(19) United States
(12) Patent Application Publication
(10) Pub. No.: US 2026/0041902 A1
(43) Pub. Date: Feb. 12, 2026

(54) PULSATILE VENTRICULAR ASSIST DEVICES

(71) Applicants: Yossi GROSS, Moshav Mazor (IL); Oz CABIRI, Hod HaSharon (IL)

(72) Inventors: Yossi GROSS, Moshav Mazor (IL); Oz CABIRI, Hod HaSharon (IL)

(21) Appl. No.: 19/173,432

(22) Filed: Apr. 8, 2025

Related U.S. Application Data
(63) Continuation-in-part of application No. 19/039,635, filed on Jan. 28, 2025, now Pat. No. 12,427,300.
(63) Continuation-in-part of application No. 18/795,359, filed on Aug. 6, 2024, now Pat. No. 12,427,299.

(51) Int. Cl. A61M 60/17; A61M 39/24; A61M 60/258; A61M 60/462; A61M 60/515; A61M 60/569

(52) U.S. Cl. CPC A61M 60/17; A61M 39/24; A61M 60/258; A61M 60/462; A61M 60/515; A61M 60/569; A61M 2205/106; A61M 2205/3303; A61M 2205/3327; A61M 2205/3331; A61M 2205/50; A61M 2206/10; A61M 2210/125; A61M 2230/005; A61M 2230/04

(57) ABSTRACT

A method is provided that includes inserting an inflow cannula of a left ventricular assist device (LVAD) of a LVAD system into a left chamber of a heart of a patient transeptally via a right atrium and a superior vena cava (SVC). An outflow cannula of the LVAD is coupled to a left subclavian artery or a left axillary artery. Other embodiments are also described.

### CROSS-REFERENCE TO RELATED APPLICATIONS

The present application claims priority from and is a continuation-in-part (CIP) of:
U.S. application Ser. No. 18/795,359, filed Aug. 6, 2024, and
U.S. application Ser. No. 19/039,635, filed Jan. 28, 2025.

All of the above-referenced applications are incorporated herein by reference.

### FIELD OF THE APPLICATION

The present invention relates generally to ventricular assist devices, and specifically to pulsatile ventricular assist devices.

### BACKGROUND OF THE APPLICATION

A left ventricular assist device (LVAD) is an electromechanical device that helps pump blood from the left ventricle to partially or to completely replace the function of a failing heart. The two main types of LVADs are pulsatile LVADs and continuous-flow LV ADs, also known as nonpulsatile LVADs. Pulsatile LV ADs mimic the natural rhythmic action of the heart, while continuous-flow LVADs use a motor at fixed velocities, leading to nearly constant pressure in the ascending aorta, i.e., minimal pulsatility.

As a consequence, continuous-flow LVADs are associated with substantial side effects, including:
altered arterial baroreceptors—reduced pulsatility leads to increased sympathetic activation and peripheral vascular resistance;
increased cardiovascular risk factors, which include essential hypertension, stroke, chronic kidney disease, and reduced likelihood of myocardial recovery;
increased aortic leaflet strain, which may cause aortic valve issues like fusion and regurgitation;
gastrointestinal bleeding, which is common due to lack of pulsatility and enhanced proteolysis;
reduced blood pressure control, which is crucial for maintaining flow and cardiac output; and
blood pressure measurement challenges, because standard methods are less effective without pulsatile flow.

In addition, continuous-flow lvad malfunction can result in death.

U.S. Pat. No. 11,565,104 to Gross describes a mechanical circulatory assist device that includes a stent, a coiled wire wound around the stent, and a reciprocating valve including a housing, one or more leaflets coupled to the housing, and one or more permanent magnets coupled to the housing. The magnets are arranged to interact with a magnetic field generated by the coiled wire when current flows therethrough, so as to axially move the reciprocating valve with respect to the stent when the reciprocating valve is disposed within the stent. Upstream axial motion of the reciprocating valve causes the leaflets to be in an open state in which they allow blood flow through the reciprocating valve. Downstream axial motion of the reciprocating valve causes the leaflets to be in a closed state in which they inhibit blood flow through the reciprocating valve. Other embodiments are also described.

U.S. Pat. No. 10,568,999 to Gross describes apparatus for deployment in a lumen of a blood vessel of a subject. The apparatus includes a reciprocating device configured to move downstream and upstream in the blood vessel in a reciprocating pattern to provide: (i) a first effective surface area of the device for pushing blood downstream in the blood vessel during downstream motion of the reciprocating device, and (ii) second effective surface area of the device during upstream motion of the reciprocating device. The first effective surface area is larger for pushing blood in the blood vessel than the second effective surface area. The apparatus further includes a device driver configured to drive the reciprocating device in the reciprocating pattern. Other applications are also described.

PCT Publication WO 2004/073484 to Gross et al. describes apparatus that includes an inflatable bladder, adapted to be coupled to a blood vessel of a subject carrying oxygenated blood, such that an interior of the bladder is in fluid communication with the blood. The apparatus also includes a piston in mechanical communication with the bladder; a motor, adapted to synchronize contraction and expansion of the bladder with a cardiac cycle of the subject by applying a motor force to the piston; and a spring, adapted to apply a spring force to the piston.

U.S. Pat. No. 5,693,091 to Larson, Jr. et al. describes, in one embodiment, a surgically implantable reciprocating pump that employs a check valve as the piston, which is driven by a permanent magnet linear electric motor to assist either side of the natural heart. The pump is implanted in the aorta or pulmonary artery using vascular attachment cuffs such as flexible cuffs for suturing at each end with the pump output directly in line with the artery. The pump is powered by surgically implanted rechargeable batteries. In another embodiment, pairs of pumps are provided to replace or assist the natural heart or to provide temporary blood flow throughout the body, for example, during operations to correct problems with the natural heart.

U.S. Pat. No. 9,555,173 to Spanier describes an extravascular pulsation blood pump that possesses a bidirectionally acting pumping system having a pump which is connected via a first conduit to the left ventricle and via a second conduit to the aorta. By means of a control means, the pump is operated alternately in one and the other direction according to a given cardiac rhythm, so that alternately blood is sucked through the first conduit and simultaneously blood ejected through the second conduit to the same extent, on the one hand, and blood is sucked through the second conduit and simultaneously blood ejected through the first conduit, on the other hand. The pulsation blood pump combines the functions and advantages of an extravascular copulsation pump with those of an extravascular counterpulsation blood pump.

### SUMMARY OF THE APPLICATION

Some embodiments of the present invention provide a left ventricular assist device (LVAD) system for treating a patient. The LVAD system comprises an implantable LVAD for implantation in the patient. The implantable LVAD comprises a pump, which is shaped so as to define a pump chamber having an upstream inflow end and a downstream outflow end. The pump comprises a tubular linear motor comprising a magnetic piston and a stator. The magnetic piston comprises a reciprocating one-way valve configured to allow downstream blood flow and inhibit upstream blood flow. The stator is configured to magnetically drive the magnetic piston with reciprocating motion, so as to pump blood downstream during downstream motion of the magnetic piston while the reciprocating one-way valve is closed. For some applications, the pump further comprises a spring, which is arranged to store energy during upstream motion of the magnetic piston and release the stored energy during the downstream motion of the magnetic piston.

The LVAD further comprises outflow and inflow cannulas. The outflow cannula is couplable in fluid communication with a circulatory system of the patient at a first site, and arranged in fluid communication with the downstream outflow end of the pump chamber via a stationary one-way outflow valve of the LVAD. The stationary one-way outflow valve is configured to allow downstream blood flow from the pump chamber to the outflow cannula and to inhibit upstream blood flow from the outflow cannula to the pump chamber. The inflow cannula is couplable in fluid communication with the circulatory system at a second site upstream of the first site, and arranged to allow downstream blood flow from the inflow cannula to the upstream inflow end of the pump chamber.

The LVAD system further comprises a cardiac sensor and control circuitry. The cardiac sensor is configured to sense one or more features of a plurality of cardiac cycles of the heart. The control circuitry is coupled to the cardiac sensor and configured to activate the tubular linear motor to provide pulsatile flow synchronized with the cardiac cycles by, during each of the cardiac cycles:
activating the stator of the tubular linear motor, during at least a portion of systole, to move the magnetic piston downstream during a first period of time, and
activating the stator of the tubular linear motor, during at least a portion of diastole, to move the magnetic piston upstream during a second period of time.

In configurations in which the pump further comprises the spring, the motion of the magnetic piston upstream during the second period stores energy in the spring, which releases the stored energy during systole, thereby reducing the amount of energy that the tubular linear motor must apply during systole in order to achieve a given amount of downstream motion of the magnetic piston. This allocation of activation of the tubular linear motor between diastole and systole reduces the peak power consumed by the tubular linear motor during systole, which may reduce the motor's demands on a power source (e.g., comprising one or more batteries) of the LVAD system. For example, the control circuitry and the spring may be configured such that, during each of the cardiac cycles, peak power consumed by the tubular linear motor during systole is no more than three times, such as no more than two times, peak power consumed by the tubular linear motor during diastole.

The activation of the tubular linear motor to pump blood downstream only during a portion of systole provides pulsatile blood flow, which mimics the healthy heart's natural function. Pulsatility in blood flow is considered crucial for normal organ function, particularly for critical organs such as the kidney, liver, brain, and heart.

In an application of the present invention, the control circuitry is configured to activate the tubular linear motor to provide pulsatile flow synchronized with the cardiac cycles by:
during at least a portion of systole of each of the cardiac cycles, activating the stator of the tubular linear motor to move the magnetic piston downstream at a velocity set based on a target stroke volume and a target stroke duration, and
during at least a portion of diastole of each of the cardiac cycles, activating the stator of the tubular linear motor to move the magnetic piston upstream.

Thus, the control circuitry uses the target stroke volume and target stroke duration as inputs to calculate the velocity of the magnetic piston, such that the magnetic piston moves during the entirety of the target stroke duration, rather than moving the magnetic piston at a predetermined rate for the amount of time necessary to provide the target stroke volume. Utilizing the entire available target stroke duration may reduce peak energy consumption and/or provide pumping that more closely mimics the natural physiological pumping.

Some embodiments of the present invention provide another LVAD system for treating a patient. This LVAD system may optionally implement any of the features of the LVAD system described hereinabove. An LVAD of this LVAD system comprises a continuous-flow pump and a pulsatile-flow pump. The continuous-flow pump comprises a first inlet in fluid communication with inflow cannula, and a first outlet. The pulsatile-flow pump comprises a second inlet, and a second outlet, which is in fluid communication with the outflow cannula. The second inlet of the pulsatile-flow pump is in fluid communication with the first outlet of the continuous-flow pump, and thus with the inflow cannula via the continuous-flow pump. The inflow cannula is therefore arranged to allow downstream blood flow from the inflow cannula to the second inlet. The pulsatile-flow pump may implement any of the features of the pulsatile-flow pump described hereinabove, mutatis mutandis.

The control circuitry of the LVAD system is configured to:
activate the continuous-flow pump to provide flow without synchronization with cardiac cycles of a heart of the patient, and
activate the pulsatile-flow pump to provide pulsatile flow synchronized with the cardiac cycles.

Some embodiments of the present invention provide yet another LVAD system for treating a patient. This LVAD system may optionally implement any of the features of the LVAD system described hereinabove.

An LVAD of the LVAD system comprises a pulsatile-flow pump, which comprises an upstream inlet and a downstream outlet; an inflow cannula, which is (1) configured to be inserted into a left ventricle of the patient via an aortic valve and an ascending aorta, and (2) arranged in fluid communication with the upstream inlet of the pulsatile-flow pump; and an outflow cannula, which is (1) couplable to an artery of the systemic circulation of the patient, and (2) arranged in fluid communication with the downstream outlet of the pulsatile-flow pump. The LVAD system typically further comprises a cardiac sensor configured to sense one or more features of a cardiac cycle of the heart; control circuitry, which is coupled to the cardiac sensor and configured to activate the pulsatile-flow pump to provide pulsatile flow synchronized with the cardiac cycle in a counterpulsation mode, by pumping blood in a downstream direction out of the downstream outlet during at least a portion of ventricular diastole; and, typically, a power source arranged to provide power to the pulsatile-flow pump.

By drawing blood out of the left ventricle during diastole, the LVAD system may reduce the ventricular filling pressure and/or reduce atrial pressure, thereby preventing congestion; reduce ventricle wall stress; and/or elevate diastolic pressure.

For some applications, an upstream end opening of the outflow cannula is arranged in the fluid communication with the downstream outlet of the pulsatile-flow pump, and a downstream end of the outflow cannula is configured to be anastomosed to the artery such that a downstream end opening of the outflow cannula is in fluid communication with the artery via an arteriotomy.

For some applications, an outer cross-sectional area of the inflow cannula is less than an inner cross-sectional area of the outflow cannula, the cross-sectional areas measured perpendicular to respective longitudinal axes of the cannulas. The outflow cannula is shaped so as to define a lateral opening. The inflow cannula is arranged passing through (a) the downstream end opening of the outflow cannula, (b) a longitudinal portion of the outflow cannula, and (c) the lateral opening of the outflow cannula, so as to (i) form a liquid-tight seal between an outer surface of the inflow cannula and the lateral opening, and (ii) allow blood flow within the outflow cannula outside and alongside the inflow cannula. The inflow cannula is configured to be positioned passing through the arteriotomy. This arrangement allows the implantation of both the outflow cannula and the inflow cannula by making only a single arteriotomy and performing only a single anastomosis, which may simplify the implantation procedure.

Some embodiments of the present invention provide a right ventricular assist device (RVAD) system, which comprises an implantable RVAD comprising:
a pulsatile-flow pump, which is shaped so as to define a pump chamber, which includes a first sub-chamber having a proximal opening, and a second sub-chamber having a distal opening;
a proximal cannula, which is (1) configured to be inserted into a right ventricle of the patient, and (2) arranged in fluid communication with the proximal opening of the first sub-chamber of the pump chamber; and
a distal cannula, which is (1) couplable to an artery of the systemic circulation of the patient, and (2) arranged in fluid communication with the distal opening of the second sub-chamber of the pump chamber.

The first and the second sub-chambers are in fluid isolation from each other, such that the proximal opening and the distal opening are in fluid isolation from each other.

The RVAD system typically further comprises a cardiac sensor, configured to sense one or more features of a cardiac cycle of the heart; control circuitry; and, typically, a power source arranged to provide power to the pulsatile-flow pump.

The control circuitry is configured to activate the pulsatile-flow pump to provide pulsatile flow synchronized with the cardiac cycle in a counterpulsation mode, by:
during at least a portion of ventricular diastole, pumping blood in a distal direction out of the distal opening of the second sub-chamber of the pump chamber and drawing blood in the distal direction into the proximal opening of the first sub-chamber of the pump chamber, and
during at least a portion of ventricular systole, pumping blood in a proximal direction out of the proximal opening of the first sub-chamber of the pump chamber and drawing blood in the proximal direction into the distal opening of the second sub-chamber of the pump chamber.

Thus, during diastole, the pulsatile-flow pump draws from the right ventricle, thereby reducing venous pressure and pushing blood to the arterial systemic circulation. During systole, the pulsatile-flow pump draws blood from the arterial systemic circulation and reduces the pressure on the left ventricle.

There is therefore provided, in accordance with an application of the present invention, a left ventricular assist device (LVAD) system for treating a patient, the LVAD system including:
(i) an implantable LVAD for implantation in the patient, the LVAD including:
   (a) an outflow cannula, which is couplable in fluid communication with a circulatory system of the patient at a first site;
   (b) an inflow cannula, which is couplable in fluid communication with the circulatory system at a second site upstream of the first site;
   (c) a continuous-flow pump, which includes (1) a first inlet in fluid communication with the inflow cannula, and (2) a first outlet; and
   (d) a pulsatile-flow pump, which includes (1) a second inlet in fluid communication with the first outlet of the continuous-flow pump, and (2) a second outlet in fluid communication with the outflow cannula; and
(ii) control circuitry, which is configured to:
   activate the continuous-flow pump to provide flow without synchronization with cardiac cycles of a heart of the patient, and
   activate the pulsatile-flow pump to provide pulsatile flow synchronized with the cardiac cycles.

For some applications, the LVAD further includes a cardiac sensor, which is configured to sense one or more features of the cardiac cycles, and the control circuitry is coupled to the cardiac sensor.

For some applications, the second site is an apex of a left ventricle of the heart.

For some applications, the LVAD further includes a stationary one-way outflow valve, which is arranged to allow downstream blood flow from the second outlet of the pulsatile-flow pump to the outflow cannula, and to inhibit upstream blood flow from the outflow cannula to the second outlet.

For some applications, the LVAD system does not include a balloon.

For some applications, the LVAD system does not include a compliance chamber.

For some applications, the LVAD further includes a tube which couples the second inlet of the pulsatile-flow pump in the fluid communication with the first outlet of the continuous-flow pump.

For any of the applications described above, the pulsatile-flow pump may:
be shaped so as to define a pump chamber having (a) an upstream inflow end in fluid communication with the second inlet, and (b) a downstream outflow end in fluid communication with the second outlet, and
include a tubular linear motor, which includes (a) a magnetic piston, which includes a reciprocating one-way valve configured to allow downstream blood flow and inhibit upstream blood flow; and (b) a stator, which is configured to magnetically drive the magnetic piston with reciprocating motion, so as to pump blood downstream during downstream motion of the magnetic piston while the reciprocating one-way valve is closed.

For some applications, the control circuitry is configured to activate the tubular linear motor to provide the pulsatile flow synchronized with the cardiac cycles by, during each of the cardiac cycles:
activating the stator of the tubular linear motor to move the magnetic piston downstream during a portion of systole of the cardiac cycle, and
activating the stator of the tubular linear motor to move the magnetic piston upstream during at least a portion of diastole of the cardiac cycle.

For some applications, the LVAD further includes a stationary one-way outflow valve, which is arranged to allow downstream blood flow from the second outlet of the pulsatile-flow pump to the outflow cannula, and to inhibit upstream blood flow from the outflow cannula to the second outlet.

For some applications, the LVAD further includes a stationary one-way inflow valve, which is arranged to allow downstream blood flow into the pump chamber of the pulsatile-flow pump, and to inhibit upstream blood flow from the pump chamber.

For some applications, the pump chamber is rigid.

For any of the applications described above, the continuous-flow pump may be a magnetically-levitated centrifugal pump.

For any of the applications described above, the control circuitry may be configured to activate the pulsatile-flow pump to provide pulsatile flow synchronized with the cardiac cycles by, during each of the cardiac cycles, activating the pulsatile-flow pump to pump blood downstream during a portion of systole of the cardiac cycle.

For some applications, the control circuitry is configured to activate the pulsatile-flow pump to pump blood downstream during the portion of systole of the cardiac cycle, and not to pump blood downstream during any portion of diastole of the cardiac cycle.

For some applications, the control circuitry is configured, during each of the cardiac cycles, to activate the pulsatile-flow pump to begin the portion of systole at a delay after the beginning of systole.

For some applications, a duration of the delay is 200-400 milliseconds, such as 300-400 milliseconds.

For some applications:
the LVAD system further includes a cardiac sensor, which is configured to sense one or more features of the cardiac cycles,
the control circuitry is coupled to the cardiac sensor, and
the control circuitry is configured to set a duration of the delay equal to 20%-40% of a total duration of the cardiac cycle.

For some applications, the control circuitry is configured to activate the pulsatile-flow pump to begin the portion of systole upon detection by the control circuitry of a beginning of a decline in pressure after a rise in pressure during systole.

For some applications:
the pulsatile-flow pump:
   is shaped so as to define a pump chamber having (a) an upstream inflow end in fluid communication with the second inlet, and (b) a downstream outflow end in fluid communication with the second outlet, and
   includes a tubular linear motor, which includes (a) a magnetic piston, which includes a reciprocating one-way valve configured to allow downstream blood flow and inhibit upstream blood flow; and (b) a stator, which is configured to magnetically drive the magnetic piston with reciprocating motion, so as to pump blood downstream during downstream motion of the magnetic piston while the reciprocating one-way valve is closed,
the LVAD system further includes an upstream pressure sensor, which is disposed and configured to measure blood pressure of blood upstream of the magnetic piston of the pulsatile-flow pump, and
the control circuitry is configured to detect the beginning of the decline in the pressure after the rise in the pressure during systole, using the upstream pressure sensor.

For some applications, the upstream pressure sensor is disposed and configured to measure the blood pressure of blood entering the upstream inflow end of the pump chamber of the pulsatile-flow pump.

For some applications, the upstream pressure sensor is disposed upstream of the continuous-flow pump, and configured to measure left-ventricular pressure (LVP).

There is further provided, in accordance with an application of the present invention, a method for treating a patient including:
implanting a left ventricular assist device (LVAD) of an LVAD system in the patient, by:
   coupling an outflow cannula of the LVAD in fluid communication with a circulatory system of the patient at a first site; and
   coupling an inflow cannula of the LVAD in fluid communication with the circulatory system at a second site upstream of the first site,
   wherein the LVAD includes (a) a continuous-flow pump, which includes (1) a first inlet in fluid communication with the inflow cannula, and (2) a first outlet, and (b) a pulsatile-flow pump, which includes (1) a second inlet in fluid communication with the first outlet of the continuous-flow pump, and (2) a second outlet in fluid communication with the outflow cannula; and activating control circuitry of the LVAD system to:
   activate the continuous-flow pump to provide flow without synchronization with cardiac cycles of a heart of the patient, and
   activate the pulsatile-flow pump to provide pulsatile flow synchronized with the cardiac cycles.

For some applications, the LVAD system further includes a cardiac sensor, which is configured to sense one or more features of the cardiac cycles, and the control circuitry is coupled to the cardiac sensor.

For some applications, the LVAD system further includes a stationary one-way outflow valve, which is arranged to allow downstream blood flow from the second outlet of the pulsatile-flow pump to the outflow cannula, and to inhibit upstream blood flow from the outflow cannula to the second outlet.

For some applications, the LVAD system further includes a tube which couples the second inlet of the pulsatile-flow pump in the fluid communication with the first outlet of the continuous-flow pump.

For some applications, the pulsatile-flow pump:
is shaped so as to define a pump chamber having (a) an upstream inflow end in fluid communication with the second inlet, and (b) a downstream outflow end in fluid communication with the second outlet, and
includes a tubular linear motor, which includes (a) a magnetic piston, which includes a reciprocating one-way valve configured to allow downstream blood flow and inhibit upstream blood flow; and (b) a stator, which is configured to magnetically drive the magnetic piston with reciprocating motion, so as to pump blood downstream during downstream motion of the magnetic piston while the reciprocating one-way valve is closed.

For some applications, activating the control circuitry includes activating the control circuitry to activate the tubular linear motor to provide the pulsatile flow synchronized with the cardiac cycles by, during each of the cardiac cycles:
activating the stator of the tubular linear motor to move the magnetic piston downstream during a portion of systole of the cardiac cycle, and
activating the stator of the tubular linear motor to move the magnetic piston upstream during at least a portion of diastole of the cardiac cycle.

For some applications, the LVAD system further includes a stationary one-way outflow valve, which is arranged to allow downstream blood flow from the second outlet of the pulsatile-flow pump to the outflow cannula, and to inhibit upstream blood flow from the outflow cannula to the second outlet.

For some applications, the LVAD system further includes a stationary one-way inflow valve, which is arranged to allow downstream blood flow into the pump chamber of the pulsatile-flow pump, and to inhibit upstream blood flow from the pump chamber.

For some applications, the continuous-flow pump includes a magnetically-levitated centrifugal pump.

For some applications, activating the control circuitry includes activating the control circuitry to activate the pulsatile-flow pump to provide pulsatile flow synchronized with the cardiac cycles by, during each of the cardiac cycles, activating the pulsatile-flow pump to pump blood downstream during a portion of systole of the cardiac cycle.

For some applications, activating the control circuitry includes activating the control circuitry to activate the pulsatile-flow pump to pump blood downstream during the portion of systole of the cardiac cycle, and not to pump blood downstream during any portion of diastole of the cardiac cycle.

For some applications, activating the control circuitry includes activating the control circuitry to, during each of the cardiac cycles, activate the pulsatile-flow pump to begin the portion of systole at a delay after the beginning of systole.

For some applications, a duration of the delay is 200-400 milliseconds, such as 300-400 milliseconds.

For some applications, the LVAD system further includes a cardiac sensor, which is configured to sense one or more features of the cardiac cycles, the control circuitry is coupled to the cardiac sensor, and activating the control circuitry includes activating the control circuitry to set a duration of the delay equal to 20%-40% of a total duration of the cardiac cycle.

For some applications, activating the control circuitry includes activating the control circuitry to activate the pulsatile-flow pump to begin the portion of systole upon detection by the control circuitry of a beginning of a decline in pressure after a rise in pressure during systole.

For Some Applications:
the pulsatile-flow pump:
   is shaped so as to define a pump chamber having (a) an upstream inflow end in fluid communication with the second inlet, and (b) a downstream outflow end in fluid communication with the second outlet, and
   includes a tubular linear motor, which includes (a) a magnetic piston, which includes a reciprocating one-way valve configured to allow downstream blood flow and inhibit upstream blood flow; and (b) a stator, which is configured to magnetically drive the magnetic piston with reciprocating motion, so as to pump blood downstream during downstream motion of the magnetic piston while the reciprocating one-way valve is closed,
the LVAD system further includes an upstream pressure sensor, which is disposed and configured to measure blood pressure of blood upstream of the magnetic piston of the pulsatile-flow pump, and
activating the control circuitry includes activating the control circuitry to detect the beginning of the decline in the pressure after the rise in the pressure during systole, using the upstream pressure sensor.

For some applications, the upstream pressure sensor is disposed and configured to measure the blood pressure of blood entering the upstream inflow end of the pump chamber of the pulsatile-flow pump.

For some applications, the upstream pressure sensor is disposed upstream of the continuous-flow pump, and configured to measure left-ventricular pressure (LVP).

For some applications, the second site is an apex of a left ventricle of the heart, and implanting the LVAD includes coupling the inflow cannula of the LVAD in fluid communication with the circulatory system at the apex of the left ventricle.

There is still further provided, in accordance with an application of the present invention, a left ventricular assist device (LVAD) system for treating a patient, the LVAD system including:
(i) an implantable LVAD for implantation in the patient, the LVAD including a pump, which (a) is shaped so as to define a pump chamber having an upstream inflow end and a downstream outflow end, and (b) includes:
   a tubular linear motor, which includes (1) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed;
   an outflow cannula, which is (1) couplable in fluid communication with a circulatory system of the patient at a first site, and (2) arranged in fluid communication with the downstream outflow end of the pump chamber; and
   an inflow cannula, which is (1) couplable in fluid communication with the circulatory system at a second site upstream of the first site, and (2) arranged to allow downstream blood flow from the inflow cannula to the upstream inflow end of the pump chamber;
(ii) a cardiac sensor, configured to sense one or more features of a plurality of cardiac cycles of a heart of the patient; and
(iii) control circuitry, which is coupled to the cardiac sensor and configured to activate the tubular linear motor to provide pulsatile flow synchronized with the cardiac cycles by, during each of the cardiac cycles:
   activating the stator of the tubular linear motor to move the magnetic piston downstream during a portion of systole of the cardiac cycle, the portion beginning at a delay after the beginning of systole, and
   activating the stator of the tubular linear motor to move the magnetic piston upstream during at least a portion of diastole of the cardiac cycle.

For some applications, a duration of the delay is 200-400 milliseconds, such as 300-400 milliseconds.

For some applications, the control circuitry is configured to set a duration of the delay equal to 20%-40% of a total duration of the cardiac cycle.

For some applications, the control circuitry is configured to activate the pulsatile-flow pump to begin the portion of systole upon detection by the control circuitry of a beginning of a decline in pressure after a rise in pressure during systole.

For some applications:
the LVAD system further includes an upstream pressure sensor, which is disposed and configured to measure blood pressure of blood upstream of the magnetic piston of the pulsatile-flow pump, and
the control circuitry is configured to detect the beginning of the decline in the pressure after the rise in the pressure during systole, using the upstream pressure sensor.

For some applications, the upstream pressure sensor is disposed and configured to measure the blood pressure of blood entering the upstream inflow end of the pump chamber of the pulsatile-flow pump.

For some applications, the upstream pressure sensor is disposed and configured to measure left-ventricular pressure (LVP).

For some applications, the LVAD system further includes a stationary one-way outflow valve, which is configured to allow downstream blood flow from the pump chamber to the outflow cannula and to inhibit upstream blood flow from the outflow cannula to the pump chamber.

There is additionally provided, in accordance with an application of the present invention, a left ventricular assist device (LVAD) system for treating a patient, the LVAD system including:
(i) an implantable LVAD for implantation in the patient, the LVAD including:
   a stationary one-way outflow valve; and
   a pump, which (a) is shaped so as to define a pump chamber having an upstream inflow end and a downstream outflow end, and (b) includes:
      a tubular linear motor, which includes (1) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed;
      a spring, which is arranged to store energy during upstream motion of the magnetic piston and release the stored energy during the downstream motion of the magnetic piston;
      an outflow cannula, which is (1) couplable in fluid communication with a circulatory system of the patient at a first site, and (2) arranged in fluid communication with the downstream outflow end of the pump chamber via the stationary one-way outflow valve, wherein the stationary one-way outflow valve is configured to allow downstream blood flow from the pump chamber to the outflow cannula and to inhibit upstream blood flow from the outflow cannula to the pump chamber; and
      an inflow cannula, which is (1) couplable in fluid communication with the circulatory system at a second site upstream of the first site, and (2) arranged to allow downstream blood flow from the inflow cannula to the upstream inflow end of the pump chamber;
(ii) a cardiac sensor, configured to sense one or more features of a plurality of cardiac cycles of a heart of the patient; and
(iii) control circuitry, which is coupled to the cardiac sensor and configured to activate the tubular linear motor to provide pulsatile flow synchronized with the cardiac cycles by, during each of the cardiac cycles:
   activating the stator of the tubular linear motor, during a first portion of the cardiac cycle, to move the magnetic piston downstream during a first period of time, and
   activating the stator of the tubular linear motor, during a second portion of the cardiac cycle, to move the magnetic piston upstream during a second period of time,
   wherein the first portion of the cardiac cycle is at least a portion of one of systole or diastole, and the second portion of the cardiac cycle is at least a portion of the other of systole or diastole.

For some applications, the first period of time has a first duration, and the second period of time has a second duration greater than the first duration.

For some applications, the spring includes an elastic spring. For some applications, the spring includes a magnetic spring.

For some applications:
the implantable LVAD further includes a stationary one-way inflow valve,
the inflow cannula is arranged to allow the downstream blood flow from the inflow cannula to the upstream inflow end of the pump chamber via the stationary one-way inflow valve, and
the stationary one-way inflow valve is configured to allow downstream blood flow from the inflow cannula to the pump chamber and to inhibit upstream blood flow from the pump chamber to the inflow cannula.

For some applications, the LVAD system further includes a battery arranged to provide power to the tubular linear motor.

For any of the applications described above, the control circuitry and the spring may be configured such that, during each of the cardiac cycles, peak power consumed by the tubular linear motor during the first portion of the cardiac cycle is no more than three times peak power consumed by the tubular linear motor during the second portion of the cardiac cycle. For some of these applications, the control circuitry and the spring are configured such that, during each of the cardiac cycles, the peak power consumed by the tubular linear motor during the first portion of the cardiac cycle is no more than two times the peak power consumed by the tubular linear motor during the second portion of the cardiac cycle.

There is yet additionally provided, in accordance with an application of the present invention, a left ventricular assist device (LVAD) system for treating a patient, the LVAD system including:
(i) an implantable LVAD for implantation in the patient, the LVAD including:
   a stationary one-way outflow valve; and
   a pump, which (a) is shaped so as to define a pump chamber having an upstream inflow end and a downstream outflow end, and (b) includes:
      a tubular linear motor, which includes (1) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed;
      an outflow cannula, which is (1) couplable in fluid communication with a circulatory system of the patient at a first site, and (2) arranged in fluid communication with the downstream outflow end of the pump chamber via the stationary one-way outflow valve, wherein the stationary one-way outflow valve is configured to allow downstream blood flow from the pump chamber to the outflow cannula and to inhibit upstream blood flow from the outflow cannula to the pump chamber; and
      an inflow cannula, which is (1) couplable in fluid communication with the circulatory system at a second site upstream of the first site, and (2) arranged to allow downstream blood flow from the inflow cannula to the upstream inflow end of the pump chamber;
(ii) a cardiac sensor, configured to sense one or more features of a plurality of cardiac cycles of a heart of the patient; and
(iii) control circuitry, which is coupled to the cardiac sensor and configured to activate the tubular linear motor to provide pulsatile flow synchronized with the cardiac cycles by:
   during a first portion of each of the cardiac cycles, activating the stator of the tubular linear motor to move the magnetic piston downstream at a velocity set based on a target stroke volume and a target stroke duration, and
   during a second portion of each of the cardiac cycles, activating the stator of the tubular linear motor to move the magnetic piston upstream,
   wherein the first portion of each of the cardiac cycles is at least a portion of one of systole or diastole, and the second portion of each of the cardiac cycles is at least a portion of the other of systole or diastole.

For some applications, the LVAD system further includes a left atrial pressure sensor, which is configured to sense left atrial pressure, and the control circuitry is configured to set the target stroke volume at a level that prevents the sensed left atrial pressure from exceeding a threshold pressure.

For some applications:
the implantable LVAD further includes a stationary one-way inflow valve,
the inflow cannula is arranged to allow the downstream blood flow from the inflow cannula to the upstream inflow end of the pump chamber via the stationary one-way inflow valve, and
the stationary one-way inflow valve is configured to allow downstream blood flow from the inflow cannula to the pump chamber and to inhibit upstream blood flow from the pump chamber to the inflow cannula.

For some applications, the LVAD system further includes a battery arranged to provide power to the tubular linear motor.

For any of the applications described above, the LVAD system may further include one or more activity sensors, which are configured to sense a level of activity of the patient, and the control circuitry may be configured to set the target stroke volume responsively to the sensed level of activity of the patient. For some applications, the one or more activity sensors include an accelerometer, which is configured to sense the level of activity of the patient. For some applications, the one or more activity sensors include a respiration sensor, which is configured to sense the level of activity of the patient by sensing one or more parameters of respiration of the patient. For some applications, the respiration sensor is configured to detect transthoracic impedance.

For any of the applications described above, the control circuitry may be configured to set the target stroke duration responsively to the sensed one or more features of the plurality of cardiac cycles. For some applications, the control circuitry is configured to set the target stroke duration responsively to a duration of systole of the plurality of cardiac cycles. For some applications, the control circuitry is configured to set the target stroke duration equal to a fraction of the duration of systole of the plurality of cardiac cycles. For some applications, the control circuitry is configured to set the target stroke duration responsively to a duration of diastole of the plurality of cardiac cycles. For some applications, the control circuitry is configured to set the target stroke duration equal to a fraction of the duration of diastole of the plurality of cardiac cycles.

There is also provided, in accordance with an application of the present invention, a left ventricular assist device (LVAD) system for treating a patient, the LVAD system including:
(i) an implantable LVAD for implantation in the patient, the LVAD including:
   a stationary one-way outflow valve; and
   a pump, which (a) is shaped so as to define a pump chamber having an upstream inflow end and a downstream outflow end, and (b) includes:
      a tubular linear motor, which includes (1) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed, wherein an inner surface of the pump chamber and
      an outer surface of the magnetic piston have respective rectangular cross-sections perpendicular to a central longitudinal axis of the pump chamber;
      an outflow cannula, which is (1) couplable in fluid communication with a circulatory system of the patient at a first site, and (2) arranged in fluid communication with the downstream outflow end of the pump chamber via the stationary one-way outflow valve, wherein the stationary one-way outflow valve is configured to allow downstream blood flow from the pump chamber to the outflow cannula and to inhibit upstream blood flow from the outflow cannula to the pump chamber; and
      an inflow cannula, which is (1) couplable in fluid communication with the circulatory system at a second site upstream of the first site, and (2) arranged to allow downstream blood flow from the inflow cannula to the upstream inflow end of the pump chamber;
(ii) a cardiac sensor, configured to sense one or more features of a plurality of cardiac cycles of a heart of the patient; and
(iii) control circuitry, which is coupled to the cardiac sensor and configured to activate the tubular linear motor to provide pulsatile flow synchronized with the cardiac cycles.

For some applications, the control circuitry is configured to activate the tubular linear motor to provide the pulsatile flow synchronized with the cardiac cycles by:
during a first portion of each of the cardiac cycles, activating the stator of the tubular linear motor to move the magnetic piston downstream,
during a second portion of each of the cardiac cycles, activating the stator of the tubular linear motor to move the magnetic piston upstream, and
the first portion of each of the cardiac cycles is at least a portion of one of systole or diastole, and the second portion of each of the cardiac cycles is at least a portion of the other of systole or diastole.

There is further provided, in accordance with an application of the present invention, a method including:
accessing a thoracic cavity of a patient; and
transthoracically implanting a left ventricular assist device (LVAD) in the patient by:
   transmurally inserting an inflow cannula of the LVAD into a left atrial appendage (LAA) of a heart, and securing the inflow cannula to a left atrial wall, and
   anastomosing an outflow cannula of the LVAD to a descending aorta.

For some applications, accessing the thoracic cavity includes performing a left thoracotomy.

For some applications:
inserting the inflow cannula into the LAA includes passing an inflow end opening of the inflow cannula through a mitral valve into a left ventricle, and
securing the inflow cannula to the left atrial wall holds the inflow end opening of the inflow cannula in the left ventricle.

For some applications, implanting the LVAD includes identifying that the patient suffers from mitral regurgitation, and passing the inflow end opening of the inflow cannula through the mitral valve into the left ventricle causes leaflets of the mitral valve to at least partially contact an outer surface of the inflow cannula during systole, thereby reducing mitral regurgitation.

For some applications, inserting the inflow cannula into the LAA includes positioning an inflow end opening of the inflow cannula in a left atrium, and securing the inflow cannula to the left atrial wall holds the inflow end opening of the inflow cannula in the left atrium.

There is still further provided, in accordance with an application of the present invention, a method including:
inserting an inflow cannula of a left ventricular assist device (LVAD) of a LVAD system into a left chamber of a heart of a patient transeptally via a right atrium and a superior vena cava (SVC); and
coupling an outflow cannula of the LVAD to an artery selected from the group consisting of: a left subclavian artery and a left axillary artery.

For some applications, inserting the inflow cannula includes inserting the inflow cannula into the left chamber of the heart transeptally via the left atrium, the SVC, and a left subclavian vein.

For some applications, inserting the inflow cannula into the left chamber of the heart includes inserting the inflow cannula into a left atrium such that an inflow end opening of the inflow cannula is within the left atrium.

For some applications, inserting the inflow cannula into the left chamber of the heart includes inserting the inflow cannula into a left ventricle via a mitral valve and a left atrium, such that an inflow end opening of the inflow cannula is within the left ventricle.

For some applications, coupling the outflow cannula to the selected artery includes anastomosing the outflow cannula to the selected artery.

For some applications, the method further includes activating control circuitry of the LVAD system to activate a pulsatile-flow pump of the LVAD to provide pulsatile flow synchronized with cardiac cycles of the patient.

For some applications:
the pulsatile-flow pump includes a pump chamber having an upstream inlet and a downstream outlet, and includes a tubular linear motor, which includes (a) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed,
the outflow cannula is arranged in fluid communication with the downstream outlet of the pump chamber,
the inflow cannula is arranged to allow downstream blood flow from the inflow cannula to the upstream inlet of the pump chamber, and
activating the control circuitry includes activating the control circuitry to activate the tubular linear motor to provide the pulsatile flow synchronized with the cardiac cycles by, during each of the cardiac cycles:
   activating the stator of the tubular linear motor to move the magnetic piston downstream during a portion of systole of the cardiac cycle, and
   activating the stator of the tubular linear motor to move the magnetic piston upstream during at least a portion of diastole of the cardiac cycle.

For some applications, the LVAD system further includes a cardiac sensor, which is configured to sense one or more features of the cardiac cycle, and the control circuitry is coupled to the cardiac sensor.

For some applications, the LVAD system further includes a stationary one-way outflow valve, which is arranged to allow downstream blood flow from the downstream outlet of the pump chamber to the outflow cannula, and to inhibit upstream blood flow from the outflow cannula to the downstream outlet.

There is additionally provided, in accordance with an application of the present invention, a left ventricular assist device (LVAD) system for treating a patient, the LVAD system including:
(i) an implantable LVAD for implantation in the patient, the LVAD including:
   (a) a pulsatile-flow pump, which includes an upstream inlet and a downstream outlet;
   (b) an inflow cannula, which is (1) configured to be inserted into a left ventricle of the patient via an aortic valve and an ascending aorta, and (2) arranged in fluid communication with the upstream inlet of the pulsatile-flow pump; and
   (c) an outflow cannula, which is (1) couplable to an artery of the systemic circulation of the patient, and (2) arranged in fluid communication with the downstream outlet of the pulsatile-flow pump;
(ii) a cardiac sensor, configured to sense one or more features of a cardiac cycle of a heart of the patient; and
(iii) control circuitry, which is configured to activate the pulsatile-flow pump to provide pulsatile flow synchronized with the cardiac cycle in a counterpulsation mode, by pumping blood in a downstream direction out of the downstream outlet during at least a portion of ventricular diastole.

For some applications, the LVAD system further includes a stationary one-way outflow valve, which is arranged to allow downstream blood flow from the downstream outlet of the pulsatile-flow pump to the outflow cannula, and to inhibit upstream blood flow from the outflow cannula to the downstream outlet.

For some applications, an outer diameter of the inflow cannula is less than an inner diameter of the outflow cannula.

For some applications, the outer diameter of the inflow cannula is less than 75% of the inner diameter of the outflow cannula.

For some applications, an outer cross-sectional area of the inflow cannula is 65%-70% of an inner cross-sectional area of the outflow cannula.

For some applications, the inflow cannula has an outer diameter of 8-13 mm.

For some applications, an upstream end opening of the outflow cannula is arranged in the fluid communication with the downstream outlet of the pulsatile-flow pump, and a downstream end of the outflow cannula is configured to be anastomosed to the artery such that a downstream end opening of the outflow cannula is in fluid communication with the artery via an arteriotomy.

For some applications:
an outer cross-sectional area of the inflow cannula is less than an inner cross-sectional area of the outflow cannula, the cross-sectional areas measured perpendicular to respective longitudinal axes of the cannulas,
the outflow cannula is shaped so as to define a lateral opening,
the inflow cannula is arranged passing through (a) the downstream end opening of the outflow cannula, (b) a longitudinal portion of the outflow cannula, and (c) the lateral opening of the outflow cannula, so as to (i) form a liquid-tight seal between an outer surface of the inflow cannula and the lateral opening, and (ii) allow blood flow within the outflow cannula outside and alongside the inflow cannula, and
the inflow cannula is configured to be positioned passing through the arteriotomy.

For some applications, the LVAD system is configured such that the inflow cannula is slidable through the lateral opening of the outflow cannula, while maintaining the liquid-tight seal between the outer surface of the inflow cannula and the lateral opening.

For some applications, (a) a volume of the outflow cannula, outside the inflow cannula, between the downstream end opening and the lateral opening of the outflow cannula equals 90%-110% of (b) a volume of the inflow cannula between the downstream end opening and the lateral opening of the outflow cannula.

For some applications, a length of the inflow cannula outside the outflow cannula, between (a) an inflow end opening of the inflow cannula and (b) the downstream end opening of the outflow cannula, is at least 20 cm.

For some applications, the pulsatile-flow pump includes:
a pump chamber, which is shaped so as to define the upstream inlet and the downstream outlet; and
a tubular linear motor, which includes (a) a magnetic piston, which includes a reciprocating one-way valve configured to allow downstream blood flow through the reciprocating one-way valve, and inhibit upstream blood flow through the reciprocating one-way valve; and (b) a stator, which is configured to magnetically drive the magnetic piston with reciprocating motion, so as to pump blood in the downstream direction out of the downstream outlet during downstream motion of the magnetic piston while the reciprocating one-way valve is closed.

For some applications, the control circuitry is configured to activate the pulsatile-flow pump to provide the pulsatile flow synchronized with the cardiac cycle in the counterpulsation mode by:
activating the stator to move the magnetic piston downstream during the at least a portion of the ventricular diastole, and
activating the stator to move the magnetic piston upstream during at least a portion of ventricular systole of the cardiac cycle.

There is yet additionally provided, in accordance with an application of the present invention, a method for treating a patient including:
implanting a left ventricular assist device (LVAD) of an LVAD system in the patient, by:
   inserting an inflow cannula of the LVAD into a left ventricle of the patient via an aortic valve and an ascending aorta, and
   coupling an outflow cannula of the LVAD to an artery of the systemic circulation of the patient,
      wherein the LVAD includes a pulsatile-flow pump, which includes (a) an upstream inlet in fluid communication with the inflow cannula, and (b) a downstream outlet in fluid communication with the outflow cannula; and
activating control circuitry of the LVAD system to activate the pulsatile-flow pump to provide pulsatile flow synchronized with a cardiac cycle of the patient in a counterpulsation mode, by pumping blood in a downstream direction out of the downstream outlet during at least a portion of ventricular diastole.

For some applications, the LVAD system further includes a cardiac sensor, which is configured to sense one or more features of the cardiac cycle, and the control circuitry is coupled to the cardiac sensor.

For some applications, the artery is selected from the group consisting of: a right axillary artery and a right subclavian artery, and coupling the outflow cannula to the artery includes coupling the outflow cannula to the selected artery.

For some applications, the LVAD system further includes a stationary one-way outflow valve, which is arranged to allow downstream blood flow from the downstream outlet of the pulsatile-flow pump to the outflow cannula, and to inhibit upstream blood flow from the outflow cannula to the downstream outlet.

For some applications, an outer diameter of the inflow cannula is less than an inner diameter of the outflow cannula.

For some applications, the outer diameter of the inflow cannula is less than 75% of the inner diameter of the outflow cannula.

For some applications, an outer cross-sectional area of the inflow cannula is 65%-70% of an inner cross-sectional area of the outflow cannula.

For some applications:
an upstream end opening of the outflow cannula is arranged in the fluid communication with the downstream outlet of the pulsatile-flow pump, and
coupling the outflow cannula to the artery includes anastomosing a downstream end of the outflow cannula to the artery such that a downstream end opening of the outflow cannula is in fluid communication with the artery via an arteriotomy.

For some applications:
an outer cross-sectional area of the inflow cannula is less than an inner cross-sectional area of the outflow cannula, the cross-sectional areas measured perpendicular to respective longitudinal axes of the cannulas,
the inflow cannula is arranged passing through (a) the downstream end opening of the outflow cannula, (b) a longitudinal portion of the outflow cannula, and (c) a lateral opening defined by the outflow cannula, so as to (i) form a liquid-tight seal between an outer surface of the inflow cannula and the lateral opening, and (ii) allow blood flow within the outflow cannula outside and alongside the inflow cannula, and
inserting the inflow cannula into the left ventricle includes inserting the inflow cannula through the arteriotomy and into the artery, and advancing the inflow cannula through the artery, the ascending aorta, and the aortic valve, into the left ventricle.

For some applications, (a) a volume of the outflow cannula, outside the inflow cannula, between the downstream end opening and the lateral opening of the outflow cannula equals 90%-110% of (b) a volume of the inflow cannula between the downstream end opening and the lateral opening of the outflow cannula.

For some applications, a length of the inflow cannula outside the outflow cannula, between (a) an inflow end opening of the inflow cannula and (b) the downstream end opening of the outflow cannula, is at least 20 cm.

For some applications:
the arteriotomy is a first arteriotomy, and
inserting the inflow cannula into the left ventricle includes:
   inserting the inflow cannula through a second arteriotomy and into the artery;
   advancing the inflow cannula through the artery, the ascending aorta, and the aortic valve, into the left ventricle; and
   anastomosing the inflow cannula to a wall of the artery around the second arteriotomy.

For some applications, the pulsatile-flow pump includes:
a pump chamber, which is shaped so as to define the upstream inlet and the downstream outlet; and
a tubular linear motor, which includes (a) a magnetic piston, which includes a reciprocating one-way valve configured to allow downstream blood flow through the reciprocating one-way valve, and inhibit upstream blood flow through the reciprocating one-way valve; and (b) a stator, which is configured to magnetically drive the magnetic piston with reciprocating motion, so as to pump blood in a downstream direction out of the downstream outlet during downstream motion of the magnetic piston while the reciprocating one-way valve is closed.

For some applications, activating the control circuitry includes activating the control circuitry to activate the pulsatile-flow pump to provide the pulsatile flow synchronized with the cardiac cycle in the counterpulsation mode by:
activating the stator to move the magnetic piston downstream during the at least a portion of the ventricular diastole, and
activating the stator to move the magnetic piston upstream during at least a portion of ventricular systole of the cardiac cycle.

There is also provided, in accordance with an application of the present invention, a ventricular assist device (VAD) system for treating a patient, the VAD system including:
(i) an implantable VAD for implantation in the patient, the VAD including:
   (a) a pump, which includes an upstream inlet and a downstream outlet;
   (b) an inflow cannula, which is (1) arranged in fluid communication with the upstream inlet of the pump, and (2) configured to be coupled to a circulatory system of the patient in fluid communication with a first site of the circulatory system; and
   (c) an outflow cannula, which has (1) an upstream end opening arranged in fluid communication with the downstream outlet of the pump, and (2) a downstream end configured to be anastomosed to an artery of the patient at a second site, such that a downstream end opening of the outflow cannula is in fluid communication with the artery via an arteriotomy; and
(ii) control circuitry, which is configured to activate the pump to provide blood flow,
an outer cross-sectional area of the inflow cannula is less than an inner cross-sectional area of the outflow cannula, the cross-sectional areas measured perpendicular to respective longitudinal axes of the cannulas,
the outflow cannula is shaped so as to define a lateral opening,
the inflow cannula is arranged passing through (a) the downstream end opening of the outflow cannula, (b) a longitudinal portion of the outflow cannula, and (c) the lateral opening of the outflow cannula, so as to (i) form a liquid-tight seal between an outer surface of the inflow cannula and the lateral opening, and (ii) allow blood flow within the outflow cannula outside and alongside the inflow cannula, and
the inflow cannula is configured to be positioned passing through the arteriotomy.

For some applications, (a) a volume of the outflow cannula, outside the inflow cannula, between the downstream end opening and the lateral opening of the outflow cannula equals 90%-110% of (b) a volume of the inflow cannula between the downstream end opening and the lateral opening of the outflow cannula.

For some applications, the LVAD system further includes a stationary one-way outflow valve, which is arranged to allow downstream blood flow from the downstream outlet of the pump to the outflow cannula, and to inhibit upstream blood flow from the outflow cannula to the downstream outlet.

For some applications, an outer diameter of the inflow cannula is less than 75% of an inner diameter of the outflow cannula.

For some applications, the outer cross-sectional area of the inflow cannula is 65%-70% of the inner cross-sectional area of the outflow cannula.

For some applications, a length of the inflow cannula outside the outflow cannula, between (a) an inflow end opening of the inflow cannula and (b) the downstream end opening of the outflow cannula, is at least 20 cm.

For some applications, the inflow cannula has an outer diameter of 8-13 mm. For some applications:
the pump includes a pulsatile-flow pump,
the implantable VAD further includes a cardiac sensor, configured to sense one or more features of a plurality of cardiac cycles of a heart of the patient, and
the control circuity is configured to activate the pulsatile-flow pump to provide pulsatile flow synchronized with the cardiac cycles.

For some applications, the pulsatile-flow pump includes:
a pump chamber, which is shaped so as to define the upstream inlet and the downstream outlet; and
a tubular linear motor, which includes (a) a magnetic piston, which includes a reciprocating one-way valve configured to allow downstream blood flow through the reciprocating one-way valve, and inhibit upstream blood flow through the reciprocating one-way valve; and (b) a stator, which is configured to magnetically drive the magnetic piston with reciprocating motion, so as to pump blood in the downstream direction out of the downstream outlet during downstream motion of the magnetic piston while the reciprocating one-way valve is closed.

There is further provided, in accordance with an application of the present invention, a method for treating a patient including:
implanting a ventricular assist device (VAD) of a VAD system in the patient, by:
   making an arteriotomy through an artery of the patient at a second site of a circulatory system of the patient;
   passing an inflow cannula of the VAD through the arteriotomy and coupling the inflow cannula to the circulatory system in fluid communication with a first site of the circulatory system, and
   anastomosing a downstream end of an outflow cannula of the VAD to the artery of the patient at the second site, such that a downstream end opening of the outflow cannula is in fluid communication with the artery via the arteriotomy,
      wherein the VAD includes a pump, which includes (a) an upstream inlet in fluid communication with the inflow cannula, and (b) a downstream outlet in fluid communication with an upstream end opening of the outflow cannula; and
activating control circuitry of the VAD system to activate the pump to provide blood flow,
wherein an outer cross-sectional area of the inflow cannula is less than an inner cross-sectional area of the outflow cannula, the cross-sectional areas measured perpendicular to respective longitudinal axes of the cannulas, and
wherein the inflow cannula is arranged passing through (a) the downstream end opening of the outflow cannula, (b) a longitudinal portion of the outflow cannula, and (c) a lateral opening defined by the outflow cannula, so as to (i) form a liquid-tight seal between an outer surface of the inflow cannula and the lateral opening, and (ii) allow blood flow within the outflow cannula outside and alongside the inflow cannula.

For some applications, the first site of the circulatory system is a left ventricle of the patient, and coupling the inflow cannula to the circulatory system in fluid communication with the first site of the circulatory system includes inserting the inflow cannula through the arteriotomy and into the artery, and advancing the inflow cannula through the artery, an ascending aorta, and an aortic valve, into the left ventricle.

For some applications, implanting the VAD includes implanting the VAD such that the first and the second sites are arterial first and second sites of the circulatory system, such that activating control circuitry causes arterial blood to flow within both the inflow cannula and the outflow cannula.

For some applications, implanting the VAD includes implanting the VAD such that the first and the second sites are venous first and second sites of the circulatory system, such that activating control circuitry causes venous blood to flow within both the inflow cannula and the outflow cannula.

For some applications, (a) a volume of the outflow cannula, outside the inflow cannula, between the downstream end opening and the lateral opening of the outflow cannula equals 90%-110% of (b) a volume of the inflow cannula between the downstream end opening and the lateral opening of the outflow cannula.

For some applications, the VAD system further includes a stationary one-way outflow valve, which is arranged to allow downstream blood flow from the downstream outlet of the pump to the outflow cannula, and to inhibit upstream blood flow from the outflow cannula to the downstream outlet.

For some applications, an outer diameter of the inflow cannula is less than an inner diameter of the outflow cannula.

For some applications, the outer diameter of the inflow cannula is less than 75% of the inner diameter of the outflow cannula.

For some applications, the outer cross-sectional area of the inflow cannula is 65%-70% of the inner cross-sectional area of the outflow cannula.

For some applications, a length of the inflow cannula outside the outflow cannula, between (a) an inflow end opening of the inflow cannula and (b) the downstream end opening of the outflow cannula, is at least 20 cm.

For some applications, the VAD further includes a cardiac sensor, configured to sense one or more features of a plurality of cardiac cycles of a heart of the patient, and the control circuitry is coupled to the cardiac sensor.

For some applications, the pump includes a pulsatile-flow pump, and the control circuity is configured to activate the pulsatile-flow pump to provide pulsatile flow synchronized with the cardiac cycles.

For some applications:
the pulsatile-flow pump includes:
a pump chamber, which is shaped so as to define the upstream inlet and the downstream outlet; and
a tubular linear motor, which includes (a) a magnetic piston, which includes a reciprocating one-way valve configured to allow downstream blood flow through the reciprocating one-way valve, and inhibit upstream blood flow through the reciprocating one-way valve; and (b) a stator, which is configured to magnetically drive the magnetic piston with reciprocating motion, so as to pump blood in a downstream direction out of the downstream outlet during downstream motion of the magnetic piston while the reciprocating one-way valve is closed.

There is still further provided, in accordance with an application of the present invention, a right ventricular assist device (RVAD) system for treating a patient, the RVAD system including:
(i) an implantable RVAD for implantation in the patient, the RVAD including:
   a pulsatile-flow pump, which is shaped so as to define a pump chamber, which includes a first sub-chamber having a proximal opening, and a second sub-chamber having a distal opening;
   a proximal cannula, which is (1) configured to be inserted into a right ventricle of the patient, and (2) arranged in fluid communication with the proximal opening of the first sub-chamber of the pump chamber; and
   a distal cannula, which is (1) couplable to an artery of the systemic circulation of the patient, and (2) arranged in fluid communication with the distal opening of the second sub-chamber of the pump chamber,
   wherein the first and the second sub-chambers are in fluid isolation from each other, such that the proximal opening and the distal opening are in fluid isolation from each other;
(ii) a cardiac sensor, configured to sense one or more features of a cardiac cycle of a heart of the patient; and
(iii) control circuitry, which is configured to activate the pulsatile-flow pump to provide pulsatile flow synchronized with the cardiac cycle in a counterpulsation mode, by:
   during at least a portion of ventricular diastole, pumping blood in a distal direction out of the distal opening of the second sub-chamber of the pump chamber and drawing blood in the distal direction into the proximal opening of the first sub-chamber of the pump chamber, and
   during at least a portion of ventricular systole, pumping blood in a proximal direction out of the proximal opening of the first sub-chamber of the pump chamber and drawing blood in the proximal direction into the distal opening of the second sub-chamber of the pump chamber.

For some applications, the proximal cannula has an outer diameter of 8-13 mm. For some applications, the proximal cannula has a length of 15-40 cm.

For some applications, the pump chamber is rigid.

For some applications, the RVAD system does not include a balloon.

For some applications, the RVAD system does not include a compliance chamber.

For some applications, the RVAD system is configured to contain blood only within the pump chamber and the proximal and the distal cannulas.

For some applications, the proximal cannula is configured to be inserted into the right ventricle via a vena cava, a right atrium, and a tricuspid valve.

For some applications, an end of the distal cannula is configured to be anastomosed to the artery.

For some applications:
the first and the second sub-chambers have first and second variable volumes, respectively, the sum of which is the volume of the pump chamber, and
the volume of the pump chamber (a) is greater than a volume of the RVAD distal to the second sub-chamber and (b) is greater than a volume of the RVAD proximal to the first sub-chamber.

For some applications, the pulsatile-flow pump includes a tubular linear motor, which includes (a) a magnetic piston slidably disposed within the pump chamber, fluidly isolating the first and the second sub-chambers from each other; and (b) a stator, which is configured to magnetically drive the magnetic piston with reciprocating motion, so as to pump blood in a distal direction during distal motion of the magnetic piston, and in a proximal direction during proximal motion of the magnetic piston.

For some applications:
a greatest cross-sectional area of the pump chamber is (a) greater than a cross-sectional area of the proximal opening of the first sub-chamber of the pump chamber and (b) greater than a cross-sectional area of the distal opening of the second sub-chamber of the pump chamber, all of the cross-sectional areas measured perpendicular to a central longitudinal axis of the pump chamber,
a proximal end wall of the first sub-chamber and a distal end wall of the second sub-chamber are frustoconical or curved, and
peripheral portions of the proximal and the distal longitudinal ends of the magnetic piston have respective shapes that correspond to the shapes of the proximal and the distal end walls, respectively.

For some applications, the control circuitry is configured to activate the pulsatile-flow pump to provide the pulsatile flow synchronized with the cardiac cycle in the counterpulsation mode, by:
during the at least a portion of ventricular diastole, activating the stator to pump blood in the distal direction out of the distal opening of the second sub-chamber of the pump chamber and to draw blood in the distal direction into the proximal opening of the first sub-chamber of the pump chamber, by moving the magnetic piston in the distal direction, and
during the at least a portion of ventricular systole, activating the stator to pump blood in the proximal direction out of the proximal opening of the first sub-chamber of the pump chamber and to draw blood in the proximal direction into the distal opening of the second sub-chamber of the pump chamber, by moving the magnetic piston in the proximal direction.

There is additionally provided, in accordance with an application of the present invention, a method for treating a patient including:
implanting a right ventricular assist device (RVAD) of an RVAD system in the patient, by (a) inserting a proximal cannula of the RVAD into a right ventricle of a heart of the patient, and (b) coupling a distal cannula of the RVAD to an artery of the systemic circulation of the patient, wherein the RVAD includes a pulsatile-flow pump, which is shaped so as to define a pump chamber, which includes (i) a first sub-chamber having a proximal opening in fluid communication with the proximal cannula, and (ii) a second sub-chamber having a distal opening in fluid communication with the distal cannula; and
activating control circuitry of the RVAD system to activate the pulsatile-flow pump to provide pulsatile flow synchronized with a cardiac cycle in a counterpulsation mode, by:
   during at least a portion of ventricular diastole, pumping blood in a distal direction out of the distal opening of the second sub-chamber of the pump chamber and drawing blood in the distal direction into the proximal opening of the first sub-chamber of the pump chamber, and
   during at least a portion of ventricular systole, pumping blood in a proximal direction out of the proximal opening of the first sub-chamber of the pump chamber and drawing blood in the proximal direction into the distal opening of the second sub-chamber of the pump chamber.

For some applications, the RVAD system further includes a cardiac sensor, which is configured to sense one or more features of the cardiac cycle, and the control circuitry is coupled to the cardiac sensor.

For some applications, the pump chamber is rigid.

For some applications, the RVAD system does not include a balloon.

For some applications, the RVAD system does not include a compliance chamber.

For some applications, the RVAD system is configured to contain blood only within the pump chamber and the proximal and the distal cannulas.

For some applications, the artery is selected from the group consisting of: a right axillary artery and a right subclavian artery, and coupling the distal cannula to the artery includes coupling the distal cannula to the selected artery.

For some applications, inserting the proximal cannula into the right ventricle includes inserting the proximal cannula into a vein and advancing the proximal cannula through the vein, a vena cava, a right atrium, and a tricuspid valve, into the right ventricle.

For some applications, coupling the distal cannula to the artery includes anastomosing an end of the distal cannula to the artery.

For some applications:
the first and the second sub-chambers have first and second variable volumes, respectively, the sum of which is the volume of the pump chamber, and
the volume of the pump chamber (a) is greater than a volume of the RVAD distal to the second sub-chamber and (b) is greater than a volume of the RVAD proximal to the first sub-chamber.

For some applications, the pulsatile-flow pump includes a tubular linear motor, which includes (a) a magnetic piston slidably disposed within the pump chamber, fluidly isolating the first and the second sub-chambers from each other; and (b) a stator, which is configured to magnetically drive the magnetic piston with reciprocating motion, so as to pump blood in the distal direction during distal motion of the magnetic piston, and in the proximal direction during proximal motion of the magnetic piston.

For some applications:
a greatest cross-sectional area of the pump chamber is (a) greater than a cross-sectional area of the proximal opening of the first sub-chamber of the pump chamber and (b) greater than a cross-sectional area of the distal opening of the second sub-chamber of the pump chamber, all of the cross-sectional areas measured perpendicular to a central longitudinal axis of the pump chamber,
a proximal end wall of the first sub-chamber and a distal end wall of the second sub-chamber are frustoconical or curved, and
peripheral portions of the proximal and the distal longitudinal ends of the magnetic piston have respective shapes that correspond to the shapes of the proximal and the distal end walls, respectively.

For some applications, activating the control circuitry includes activating the control circuitry to activate the pulsatile-flow pump to provide the pulsatile flow synchronized with the cardiac cycle in the counterpulsation mode, by:
during the at least a portion of ventricular diastole, activating the stator to pump blood in the distal direction out of the distal opening of the second sub-chamber of the pump chamber and to draw blood in the distal direction into the proximal opening of the first sub-chamber of the pump chamber, by moving the magnetic piston in the distal direction, and
during the at least a portion of ventricular systole, activating the stator to pump blood in the proximal direction out of the proximal opening of the first sub-chamber of the pump chamber and to draw blood in the proximal direction into the distal opening of the second sub-chamber of the pump chamber, by moving the magnetic piston in the proximal direction.

There is yet additionally provided, in accordance with an application of the present invention, a ventricular assist device (VAD) system for treating a patient, the VAD system including:
(i) an implantable VAD for implantation in the patient, the VAD including:
   a pump, which (a) is shaped so as to define a pump chamber having an upstream inflow end and a downstream outflow end, and (b) includes a tubular linear motor, which includes (1) a magnetic piston, and (2) a stator, which is configured to magnetically drive the magnetic piston with reciprocating motion, so as to pump blood downstream during downstream motion of the magnetic piston;
   an outflow cannula, which is (1) couplable in fluid communication with a circulatory system of the patient at a first site, and (2) arranged in fluid communication with the downstream outflow end of the pump chamber; and
   an inflow cannula, which is (1) couplable in fluid communication with the circulatory system at a second site upstream of the first site, and (2) arranged to allow downstream blood flow from the inflow cannula to the upstream inflow end of the pump chamber,
   wherein an inner surface of the pump chamber and an outer surface of the magnetic piston have respective elongate non-circular cross-sections perpendicular to a central longitudinal axis of the pump chamber,
   wherein each of the cross-sections has a first greatest dimension in one direction that is greater than a second greatest dimension in a perpendicular direction;
(ii) a cardiac sensor, configured to sense one or more features of a plurality of cardiac cycles of a heart of the patient; and
(iii) control circuitry, which is coupled to the cardiac sensor and configured to activate the tubular linear motor to provide pulsatile flow synchronized with the cardiac cycles.

For some applications, the first greatest dimension is at least 1.5 times the second greatest dimension.

For some applications, the elongate non-circular cross-sections are rectangular or elliptical.

For some applications, the elongate non-circular cross-sections are stadium-shaped.

For some applications, the elongate non-circular cross-sections respectively include two straight portions opposite each other and parallel with the first greatest dimension.

### For Some Applications:

the VAD system further includes a stationary one-way outflow valve, and the outflow cannula is arranged in the fluid communication with the downstream outflow end of the pump chamber via the stationary one-way outflow valve,
the magnetic piston includes a reciprocating one-way valve configured to allow downstream blood flow and inhibit upstream blood flow,
the stator is configured to magnetically drive the magnetic piston with the reciprocating motion, so as to pump blood downstream during the downstream motion of the magnetic piston while the reciprocating one-way valve is closed, and
the stationary one-way outflow valve is configured to allow downstream blood flow from the pump chamber to the outflow cannula and to inhibit upstream blood flow from the outflow cannula to the pump chamber.

For some applications:
the central longitudinal axis is a pump-chamber central longitudinal axis,
the reciprocating one-way valve has a valve central longitudinal axis passing through the reciprocating one-way valve, and
the valve central longitudinal axis defines an angle of at least 15 degrees with (a) the pump-chamber central longitudinal axis or (b) a line parallel to the pump-chamber central longitudinal axis.

For some applications, the angle is at least 30 degrees. For some applications, the angle is no more than 60 degrees.

For some applications, the reciprocating one-way valve includes an annulus and two or more leaflets coupled to the annulus, and the leaflets are configured to coapt with one another to close the reciprocating one-way valve, thereby inhibiting the upstream blood flow from the outflow cannula to the pump chamber.

For some applications, the stator is configured to magnetically drive the magnetic piston with the reciprocating motion, so as to pump blood downstream during the downstream motion of the magnetic piston, and upstream during upstream motion of the magnetic piston.

For some applications, the control circuitry is configured to activate the tubular linear motor to provide the pulsatile flow synchronized with the cardiac cycles by:
during a first portion of each of the cardiac cycles, activating the stator of the tubular linear motor to move the magnetic piston downstream,
during a second portion of each of the cardiac cycles, activating the stator of the tubular linear motor to move the magnetic piston upstream, and
the first portion of each of the cardiac cycles is at least a portion of one of systole or diastole, and the second portion of each of the cardiac cycles is at least a portion of the other of systole or diastole.

The present invention will be more fully understood from the following detailed description of embodiments thereof, taken together with the drawings, in which:

### BRIEF DESCRIPTION OF THE DRAWINGS

FIG. 1 is a schematic cross-sectional illustration of a left ventricular assist device (LVAD) system applied to a patient, in accordance with an application of the present invention;

FIG. 2 is a schematic illustration of an implantable LVAD of the LVAD system of FIG. 1, in accordance with an application of the present invention;

FIGS. 3A-B are schematic cross-sectional illustrations of alternative configurations of the implantable LVAD of FIG. 2, in accordance with respective applications of the present invention;

FIGS. 4A-E are schematic cross-sectional illustrations of yet additional configurations of the implantable LVAD of FIG. 2, in accordance with respective applications of the present invention;

FIG. 5A is a schematic illustration of a conventional method of coupling an implantable LVAD in fluid communication with a circulatory system of a patient;

FIGS. 5B-E are schematic illustrations of methods of coupling an implantable LVAD in fluid communication with a circulatory system of a patient, in accordance with respective applications of the present invention;

FIGS. 6A-B are schematic illustrations of another LVAD applied to a patient, in accordance with an application of the present invention;

FIG. 7 is an illustrative pump curve for a continuous-flow magnetically levitated centrifugal pump of a conventional LVAD, as known in the prior art;

FIG. 8A is an illustrative graph showing blood pressure and blood flow rate for a continuous-flow magnetically levitated centrifugal pump of a conventional LVAD, as known in the prior art;

FIGS. 8B and 8C are illustrative graphs showing blood pressure and blood flow rate for the LVAD of FIGS. 6A-B, in accordance with respective applications of the present invention;

FIGS. 9A and 9B are schematic cross-sectional illustrations of yet another LVAD system applied to a patient, in accordance with respective applications of the present invention;

FIGS. 10A and 10B are schematic cross-sectional illustrations of a right ventricular assist device (RVAD) system, in accordance with respective applications of the present invention;

FIG. 11 is a schematic cross-sectional illustration of the RVAD system of FIG. 10A applied to a patient, in accordance with an application of the present invention;

FIGS. 12A-B are schematic cross-sectional illustrations of operation of the RVAD system of FIG. 10A when applied to a patient, in accordance with an application of the present invention;

FIGS. 13A-B are schematic cross-sectional illustrations of a configuration of the implantable RVAD of FIG. 10A, in accordance with an application of the present invention; and

FIGS. 14A-B are schematic cross-sectional illustrations of another configuration of the implantable RVAD of FIG. 10A, in accordance with an application of the present invention.

### DETAILED DESCRIPTION OF APPLICATIONS

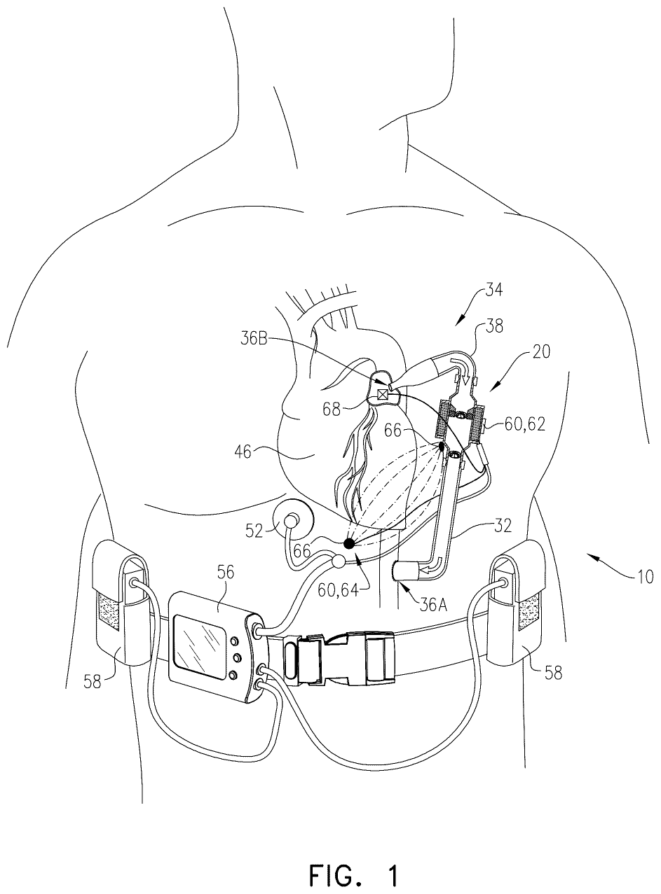

FIG. 1 is a schematic cross-sectional illustration of a left ventricular assist device (LVAD) system 10 applied to a patient, in accordance with an application of the present invention.

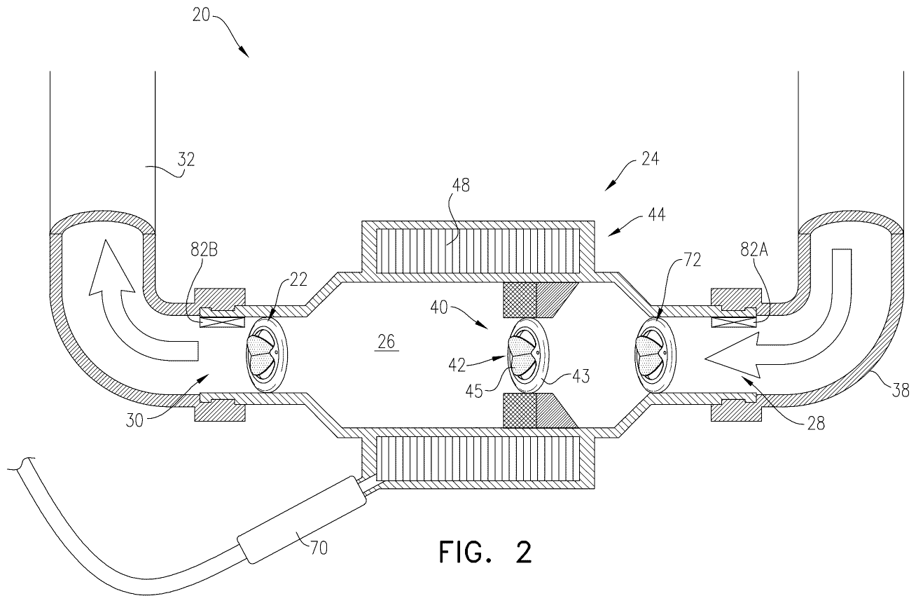

FIG. 2 is a schematic illustration of an implantable LVAD 20 of LVAD system 10, in accordance with an application of the present invention.

LVAD 20 comprises:
a stationary one-way outflow valve 22;
a pump 24, which (a) is shaped so as to define a pump chamber 26 having an upstream inflow end 28 and a downstream outflow end 30;
an outflow cannula 32, which is (a) couplable in fluid communication with a circulatory system 34 of the patient at a first site 36A, and (b) arranged in fluid communication with downstream outflow end 30 of pump chamber 26 via stationary one-way outflow valve 22; stationary one-way outflow valve 22 is configured to allow downstream blood flow from pump chamber 26 to outflow cannula 32 and to inhibit upstream blood flow from outflow cannula 32 to pump chamber 26; and
an inflow cannula 38, which is (a) couplable in fluid communication with circulatory system 34 at a second site 36B upstream of first site 36A, and (b) arranged to allow downstream blood flow from inflow cannula 38 to upstream inflow end 28 of pump chamber 26 (e.g., is arranged in fluid communication with upstream inflow end 28 of pump chamber 26).

Pump 24 is a positive displacement (PD) pump, and typically comprises a tubular linear motor 44, which comprises:
a magnetic piston 40, which comprises a reciprocating one-way valve 42 configured to allow downstream blood flow and inhibit upstream blood flow (as can be seen in the figures, reciprocating one-way valve 42 is slidably disposed within pump chamber 26); and
a stator 48, which is configured to magnetically drive magnetic piston 40 with reciprocating motion (such that magnetic piston 40 serves as the slider of tubular linear motor 44), so as to pump blood downstream during downstream motion of magnetic piston 40 while reciprocating one-way valve 42 is closed.

Stator 48 comprises coil windings, as is known in the tubular linear motor art. Tubular linear motor 44 is typically configured a levitating linear motor, in the sense that magnetic piston 40 slides within stator 48 without bearings, i.e., tubular linear motor 44 is bearingless. The use of magnetic piston 40 allows for a pump that is entirely closed, without the need for any elements external to the pump to be connected to the piston.

LVAD 20 typically generates laminar blood flow without turbulence, which results in low shear stress on red blood cells, thereby reducing the likelihood of hemolysis and thrombosis.

For some applications, such as shown in the FIGS. 1-6B and 9A-B, and labeled in FIG. 2, reciprocating one-way valve 42 comprises an annulus 43 and two or more leaflets 45 coupled to annulus 43 (e.g., exactly two or exactly three leaflets 45). Leaflets 45 are configured to coapt with one another to close reciprocating one-way valve 42, thereby inhibiting upstream blood flow. Alternatively, reciprocating one-way valve 42 comprises another type of valve known in the art, such as a duckbill valve.

LVAD system 10 typically further comprises:
a cardiac sensor 52, configured to sense one or more features of a plurality of cardiac cycles of a heart 46;
control circuitry 56, which is coupled to cardiac sensor 52 and configured to activate tubular linear motor 44 to provide pulsatile flow synchronized with the cardiac cycles; and
typically, a power source 58 (e.g., comprising one or more batteries) arranged to provide power to tubular linear motor 44.

Typically, cardiac sensor 52 comprises one or more implantable or external ECG electrodes, which are configured to sense components of an ECG of the patient. Other components of cardiac sensor 52, such as electronics, may be located either within LVAD 20 or in an external component of LVAD system 10, such as circuitry 56.

For some applications, at least a portion of control circuitry 56 and/or power source 58 are configured to be placed outside the patient's body. For these applications, LVAD system 10 may further comprise a percutaneous lead 70, which couples control circuitry 56 to LVAD 20 and/or power source 58. For example, percutaneous lead 70 may have a small diameter, e.g., 2 mm, which may reduce the risk of infection. Alternatively, control circuitry 56 and/or power source 58 may be wirelessly coupled to LVAD 20.

For some applications, at least a portion of an internal surface of pump chamber 26 is coated with a diamond-like carbon (DLC) coating.

Typically, pump chamber 26 is rigid.

Typically, the blood-contacting surfaces of LVAD 20 comprise bioprosthetic materials.

In an application of the present invention, LVAD system 10 comprises one or more activity sensors 60, which are configured to sense a level of activity of the patient. For example, the one or more activity sensors 60 may comprise one or more of the following sensors:
an accelerometer 62, which is configured to sense the level of activity of the patient by sensing motion of the patient, and/or
a respiration sensor 64, which is configured to sense the level of activity of the patient by sensing one or more parameters of respiration of the patient; for example, respiration sensor 64 may measure changes in respiration rate and/or lung volume based on transthoracic impedance, and, to this end, may comprise at least two electrodes 66 (which are typically implantable) between which impedance of one or both lungs is measured.

In an application of the present invention, control circuitry 56 is configured to adapt the stroke volume provided by tubular linear motor 44 according the metabolic demands, as indicated by the level of activity of the patient sensed using the one or more activity sensors 60. This may mimic to some extent the healthy heart, in which the stroke volume varies according to metabolic demands (in accordance with the Frank-Starling law).

In some applications of the present invention, control circuitry 56 is configured to activate tubular linear motor 44 to provide pulsatile flow synchronized with the cardiac cycles by, during each of the cardiac cycles:
activating stator 48, during a first portion of the cardiac cycle, to move the magnetic piston downstream during a first period of time, and
activating stator 48, during a second portion of the cardiac cycle, to move the magnetic piston upstream during a second period of time.

The first portion of the cardiac cycle is at least a portion of one of systole or diastole, and the second portion of the cardiac cycle is at least a portion of the other of systole or diastole.

As used in the present application, including in the claims, systole refers to ventricular systole, and diastole refers to ventricular diastole.

In an application of the present invention, control circuitry 56 is configured to activate tubular linear motor 44 to provide pulsatile flow synchronized with the cardiac cycles by:
during at least a portion of systole of each of the cardiac cycles, activating stator 48 to move magnetic piston 40 downstream at a velocity set based on a target stroke volume and a target stroke duration, and
during at least a portion of diastole of each of the cardiac cycles, activating stator 48 to move magnetic piston 40 upstream.

Thus, control circuitry 56 uses the target stroke volume and target stroke duration as inputs to calculate (e.g., mathematically or using a lookup table) the velocity of magnetic piston 40, such that magnetic piston 40 moves during the entirety of the target stroke duration, rather than moving the magnetic piston at a predetermined rate for the amount of time necessary to provide the target stroke volume. Utilizing the entire available target stroke duration may reduce peak energy consumption and/or provide pumping that more closely mimics the natural physiological pumping.

Typically, LVAD 20 is configurable to provide a stroke volume of 1-90 cc per cardiac cycle. LVAD 20 controls the provided stroke volume by setting a distance that magnetic piston 40 moves during pumping during each cardiac cycle. Typically, LVAD 20 is configured to set the stroke volume to less than a healthy heart's natural stroke volume (e.g., less than 80%-90% of a healthy heart's natural stroke volume). For some applications, control circuitry 56 is configured to allow the target stroke volume to be manually programmed by a physician.

For some applications, control circuitry 56 is configured to set the target stroke volume responsively to the level of activity of the patient sensed using one or more activity sensors 60.

For some applications, control circuitry 56 is configured to set the target stroke duration responsively to the sensed one or more features of the plurality of cardiac cycles, such as responsively to a duration of systole of the plurality of cardiac cycles, e.g., equal to a fraction of the duration of systole of the plurality of cardiac cycles.

For some applications, LVAD system 10 further comprises a left atrial pressure sensor 68, which is configured to sense left atrial pressure. Control circuitry 56 is configured to set the target stroke volume at a level that prevents the sensed left atrial pressure from exceeding a threshold pressure. For example, the threshold pressure may be 12-15 mmHg.

Reference is again made to FIG. 2. For some applications, LVAD system 10 further comprises an upstream pressure sensor 82A and/or a downstream pressure sensor 82B, which are configured to measure blood pressure of blood entering upstream inflow end 28 of pump chamber 26 and blood exiting downstream outflow end 30 of pump chamber 26, respectively. For some applications, LVAD system 10 is configured to:
detect a malfunction and/or blockage of pump 24 based on a difference between the pressures sensed by upstream and downstream pressure sensors 82A and 82B, in which case LVAD system 10 typically ceases pumping and generates an alert to the patient and/or a healthcare provider,
reduce the stroke volume if the pressure sensed by downstream pressure sensor 82B exceeds a threshold value (e.g., 120 mmHg), which may be harmful for the patient, and/or
reduce the stroke volume if the pressure sensed by upstream pressure sensor 82A falls below a threshold value, because insufficient inflow blood is available to provide the desired stroke volume.

Alternatively or additionally, LVAD system 10 may further comprise a flow sensor, which is configured to measure a velocity and/or volume of blood flow through pump 24.

For some applications, LVAD system 10 is configured to reduce the stroke volume if the current consumed by the tubular linear motor 44 exceeds a threshold value (typically measured in mA); the threshold value typically varies based on the stroke volume (e.g., based on a graph). The stroke volume is typically reduced until the current consumption falls below the threshold value for the reduced stroke volume. This feature may prevent tubular linear motor 44 from consuming more current than can or should be provided by the one or more batteries of the system.

Reference is made to FIG. 2. For some applications, implantable LVAD 20 further comprises a stationary one-way inflow valve 72. Inflow cannula 38 is arranged to allow the downstream blood flow from inflow cannula 38 to upstream inflow end 28 of pump chamber 26 via stationary one-way inflow valve 72 (e.g., is arranged in fluid communication with upstream inflow end 28 of pump chamber 26 via stationary one-way inflow valve 72). Stationary one-way inflow valve 72 is configured to allow downstream blood flow from inflow cannula 38 to pump chamber 26 and to inhibit upstream blood flow from pump chamber 26 to inflow cannula 38. Stationary one-way inflow valve 72 may prevent upstream propagation of any shock waves that may be generated by upstream motion of magnetic piston 40. Alternatively, as shown in the other figures, implantable LV AD 20 does not comprise stationary one-way inflow valve 72.

Reference is made to FIGS. 1 and 2. Typically, LVAD system 10 does comprise a balloon. Typically, LVAD system 10 does comprise a compliance chamber.

Reference is still made to FIGS. 1 and 2. Typically, LVAD system 10 is configured to contain blood only within the pump chamber 26, inflow cannula 38, and outflow cannula 32.

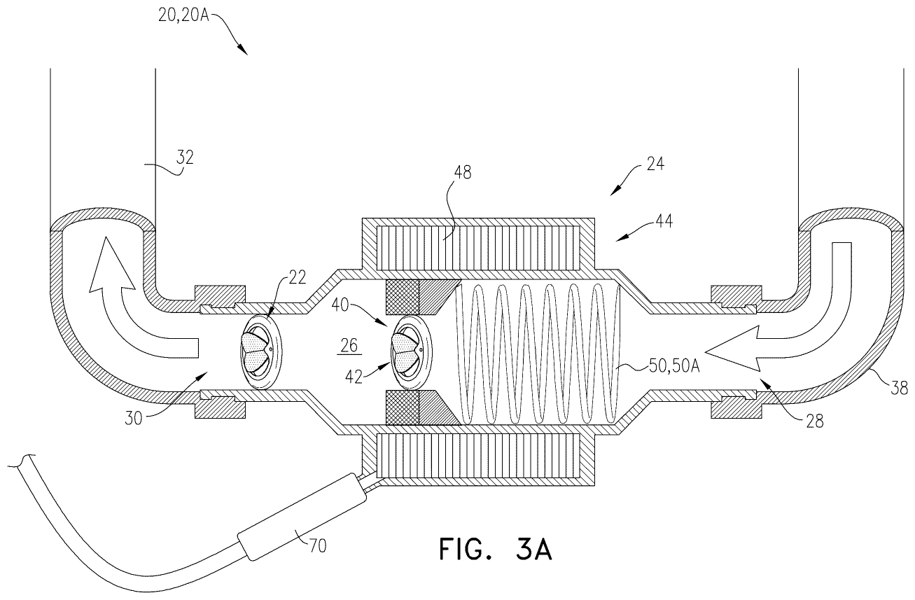

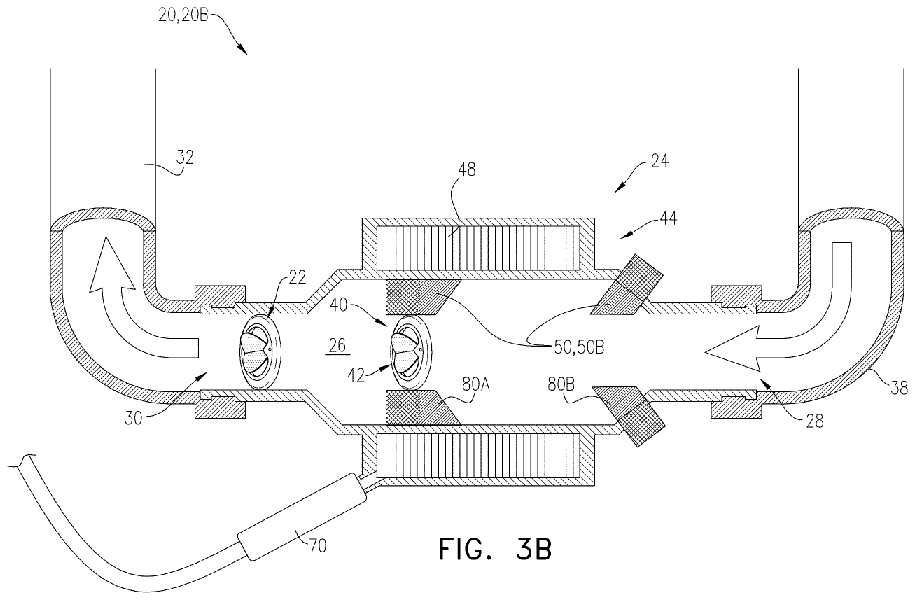

Reference is now made to FIGS. 3A-B, which are schematic cross-sectional illustrations of alternative configurations of implantable LVAD 20, in accordance with respective applications of the present invention.

In these configurations of LVAD 20, pump 24 further comprises a spring 50, which is arranged to store energy during upstream motion of magnetic piston 40 and release the stored energy during the downstream motion of magnetic piston 40. Control circuitry 56 is configured to activate tubular linear motor 44 to provide pulsatile flow synchronized with the cardiac cycles by, during each of the cardiac cycles:
activating stator 48, during at least a portion of systole, to move magnetic piston 40 downstream during a first period of time having a first duration, and
activating stator 48, during at least a portion of diastole, to move magnetic piston 40 upstream during a second period of time having a second duration.

The motion of magnetic piston 40 upstream during the second period stores energy in spring 50. Spring 50 releases the stored energy during systole, thereby reducing the amount of energy that tubular linear motor 44 must apply during systole in order to achieve a given amount of downstream motion of magnetic piston 40. This allocation of activation of tubular linear motor 44 between diastole and systole reduces the peak power consumed by tubular linear motor 44 during systole, which may reduce the motor's demands on power source 58 (which, as mentioned above, may comprise one or more batteries). For example, control circuitry 56 and spring 50 may be configured such that, during each of the cardiac cycles, peak power consumed by tubular linear motor 44 during systole is no more than three times, such as no more than two times, peak power consumed by tubular linear motor 44 during diastole.

For some applications, the second duration is greater than the first duration.

For some applications, such as shown in FIG. 3A, LVAD 20 comprises an implantable LVAD 20A, and spring 50 comprises an elastic spring 50A. For example, spring 50A may comprise a compression spring (as shown) or an extension spring (configuration not shown), or any other type of spring known in the mechanical arts. Spring 50A is coupled between magnetic piston 40 and a location fixed with respect to pump chamber 26.

For other applications, such as shown in FIG. 3B, LVAD 20 comprises an implantable LVAD 20B, and spring 50 comprises a magnetic spring 50B, which comprises a first moveable magnet 80A and a second stationary magnet 80B. Magnetic piston 40 comprises first moveable magnet 80A. First moveable magnet 80A may be one of the magnets of magnetic piston 40 used for the motorized motion of the magnetic piston, such as shown, or may be a separate magnet coupled to magnetic piston 40. Second stationary magnet 80B is coupled to pump 24 at a fixed location with respect to pump chamber 26, such as at a location axially upstream of magnetic piston 40, e.g., axially between magnetic piston 40 and upstream inflow end 28 of pump chamber 26. For example, the respective magnetic poles of first and second magnets 80A and 80B may be oriented in opposite directions, such that poles having the same polarity face each other.

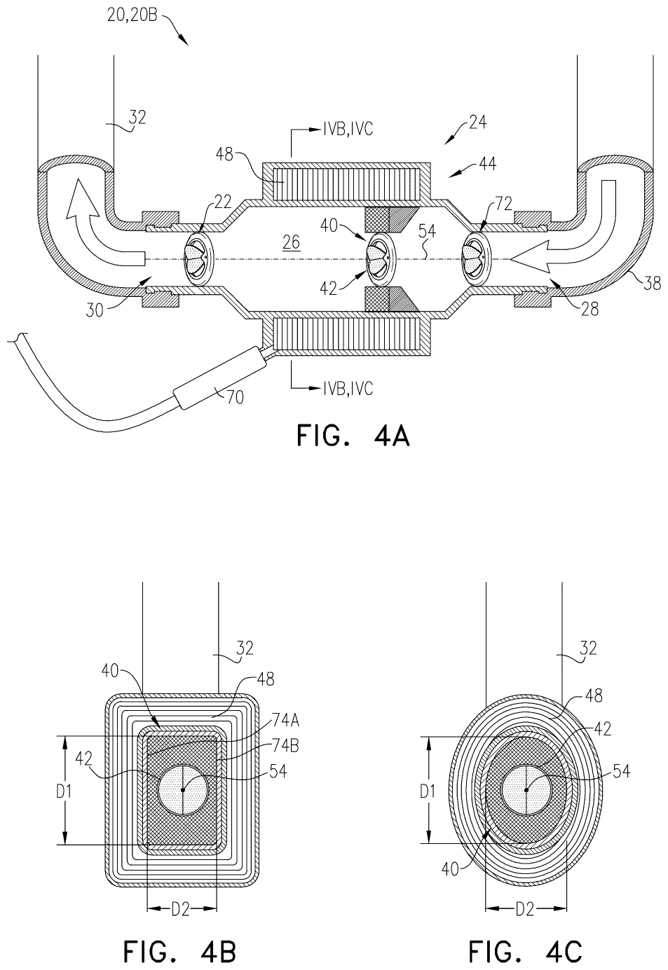

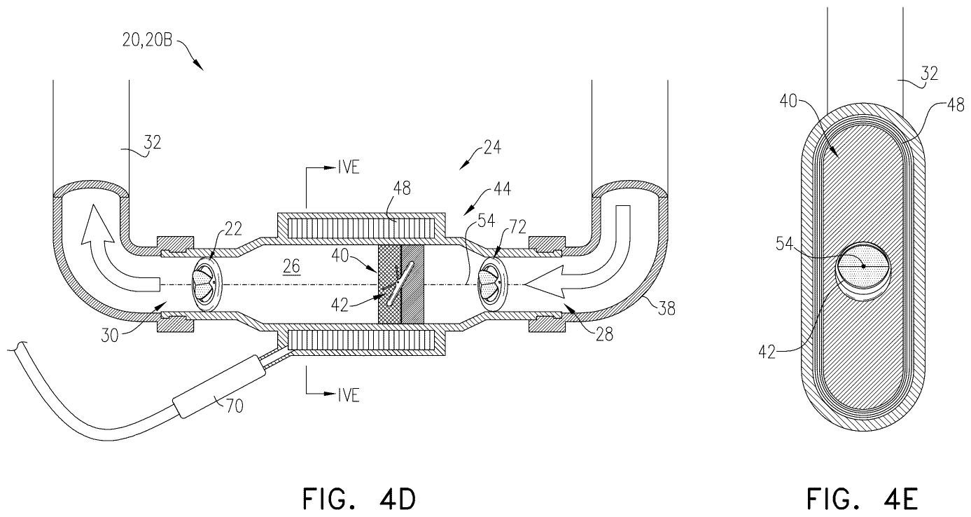

Reference is now made to FIGS. 4A-E, which are schematic cross-sectional illustrations of yet additional configurations of implantable LVAD 20, in accordance with respective applications of the present invention. The cross-sectional views of FIGS. 4B and 4C are taken along line IVB-IVB and line IVC-IVC of FIG. 4A, respectively, and the cross-sectional view of FIG. 4E is taken along line IVE-IVE of FIG. 4D. The features of these configurations may be implemented in combination with the features of any of the other configurations of implantable LVAD 20 described herein, mutatis mutandis, and like reference numerals refer to like parts.

In these configurations, an inner surface of pump chamber 26 and an outer surface of magnetic piston 40 of pump 24 have respective elongate non-circular cross-sections perpendicular to a central longitudinal axis 54 of pump chamber 26, each of which cross-sections has a first greatest dimension D1 in one direction that is greater than a second greatest dimension D2 in a perpendicular direction. For example, the first greatest dimension DI may be at least 1.5 times the second greatest dimension D2, such as at least 2 times, e.g., at least 2.5 times the second greatest dimension D2. For example, the elongate non-circular cross-sections may be rectangular, as shown in FIG. 4B; elliptical, as shown in FIG. 4C; or stadium-shaped, as shown in FIG. 4E. Optionally, the cross-sections respectively include two straight portions 74A and 74B opposite each other and parallel with the first greatest dimension D1, such as shown in FIGS. 4B and 4E. The elongate cross-sections provide a lower profile than a circular configuration, which may facilitate subcutaneous implantation. For example, rectangular cross-sections may provide a more accurate mechanism than an elliptical cross-sectional shape, because the outer surface of magnetic piston 40 may experience less friction with the inner surface of pump chamber 26 and be less likely to jam.

Reference is made to FIGS. 4D and 4E. For some applications, central longitudinal axis 54 is a pump-chamber central longitudinal axis 54, and the reciprocating one-way valve 42 has a valve central longitudinal axis 55 passing through reciprocating one-way valve 42. Valve central longitudinal axis 55 defines an angle a (alpha) of at least 15 degrees, such as at least 30 degrees (e.g., 45 degrees), with (a) pump-chamber central longitudinal axis 54 (if the axes intersect) or (b) a line parallel to pump-chamber central longitudinal axis 54 (if the axes do not intersect). Optionally, angle a (alpha) is no more than 60 degrees.

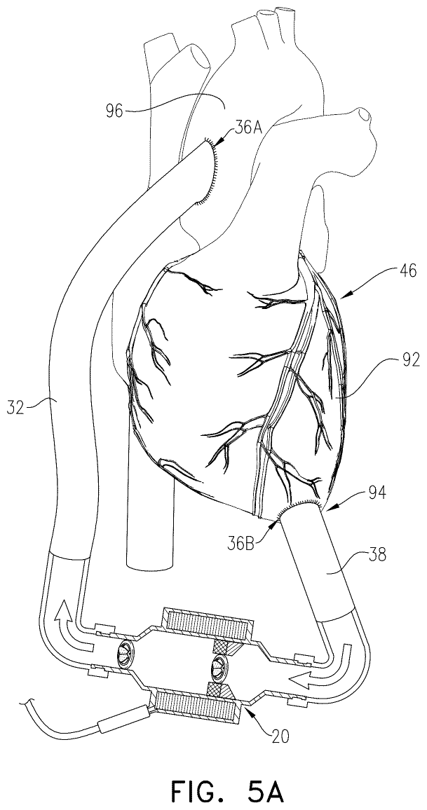

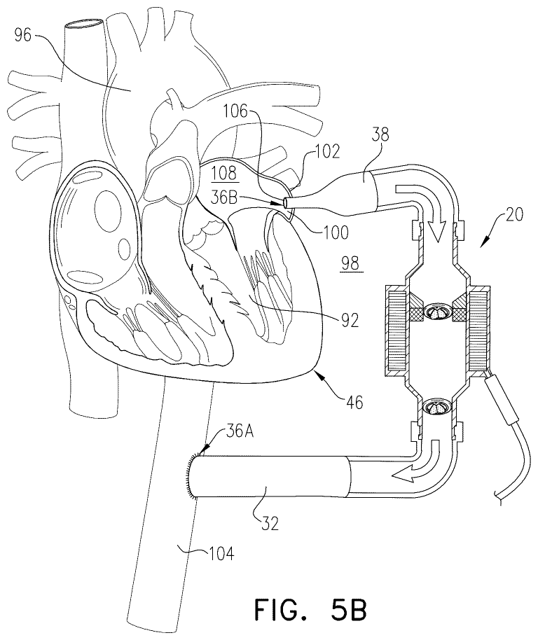

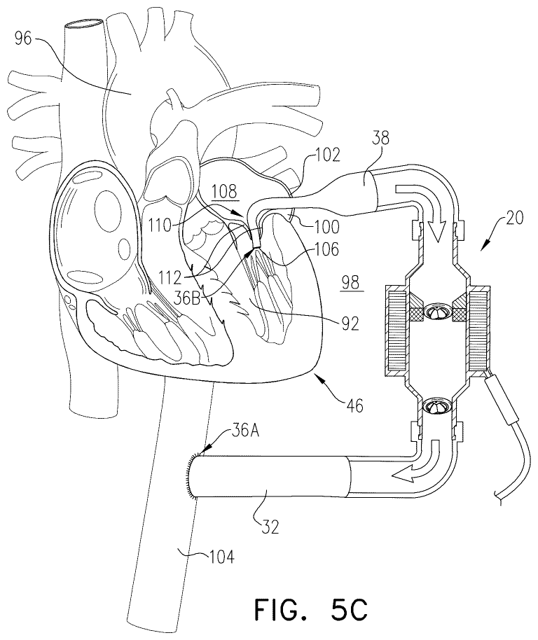

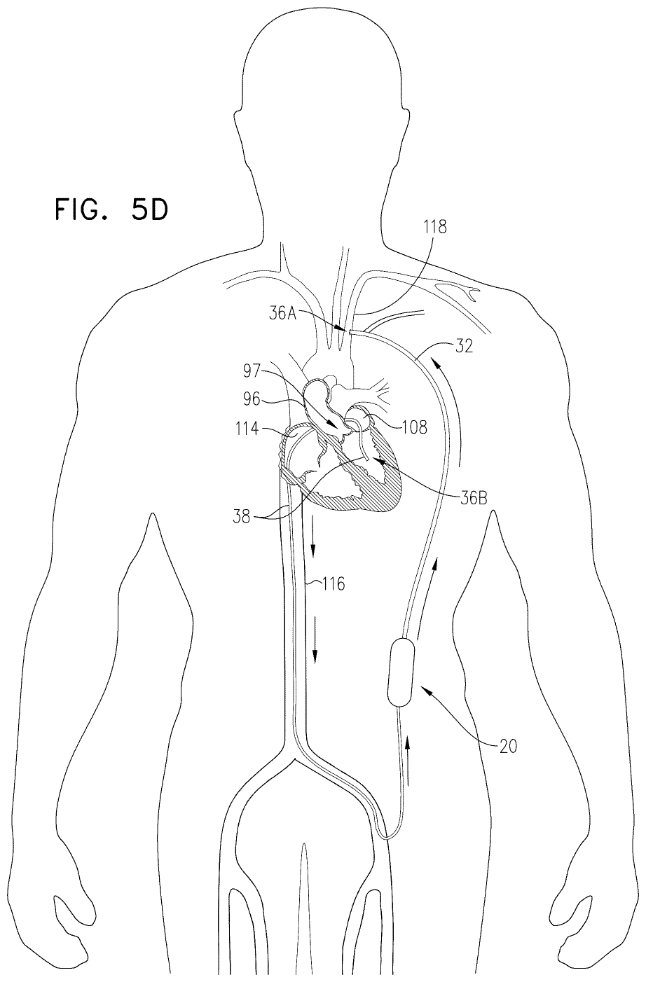

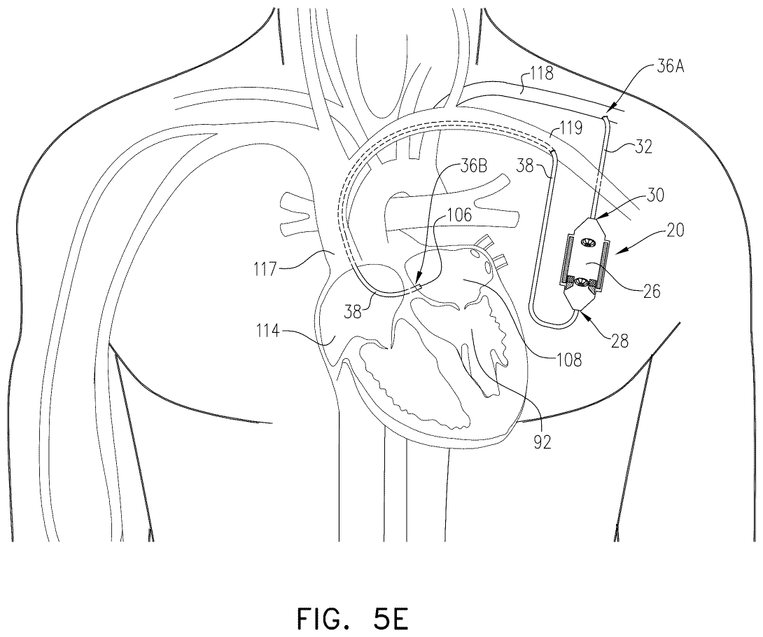

Reference is now made to FIGS. 5A-E, which are schematic illustrations of methods of coupling an implantable LVAD in fluid communication with circulatory system 34.

FIG. 5A shows a conventional coupling technique, in which inflow cannula 38 is inserted into a left ventricle 92 at the left ventricle's apex 94, and outflow cannula 32 is coupled to an ascending aorta 96 at first site 36A. In this conventional technique, a thoracic cavity 98 of the patient is accessed, typically by performing a sternotomy, which is commonly used for implanting LVADs, in order to provide access to both the left ventricle's apex 94 and ascending aorta 96.

FIGS. 5B and 5C show additional coupling techniques, in accordance with respective applications of the present invention. In these techniques, thoracic cavity 98 of the patient is accessed, typically by performing a left thoracotomy. As is known in the general surgical arts, a left thoracotomy is generally less traumatic than the sternotomy commonly used in conventional LVAD implantation techniques.

After accessing thoracic cavity 98, an LVAD (either LVAD 20 or an LVAD known in the art) is transthoracically implanted in the patient by:
transmurally inserting an inflow cannula of the LVAD into a left atrial appendage (LAA) 100 of heart 46, and securing the inflow cannula to a left atrial wall 102 (such as by stitching around the inflow cannula on an external surface of left atrial wall 102), and
anastomosing an outflow cannula of the LVAD to a descending aorta 104 at first site 36A.

Generally, it is easier to attach the outflow cannula to descending aorta 104 than ascending aorta 96, because of the easier access available to the descending aorta than to the ascending aorta.

In the technique shown in FIG. 5B, an inflow end opening 106 of the inflow cannula is positioned in a left atrium 108 (either in LAA 100 or outside LAA 100). Securing the inflow cannula to left atrial wall 102 holds inflow end opening 106 of the inflow cannula in left atrium 108.

In the technique shown in FIG. 5C, inserting the inflow cannula into LAA 100 comprises passing inflow end opening 106 of the inflow cannula through a mitral valve 110 into left ventricle 92. Securing the inflow cannula to left atrial wall 102 holds inflow end opening 106 of the inflow cannula in left ventricle 92.

For some applications, in the technique shown in FIG. 5C, implanting the LVAD comprises identifying that the patient suffers from mitral regurgitation, and passing inflow end opening 106 of the inflow cannula through mitral valve 110 into left ventricle 92 causes leaflets 112 of the mitral valve to at least partially contact an outer surface of the inflow cannula during systole, thereby reducing mitral regurgitation.

FIG. 5D shows another coupling technique, in accordance with respective applications of the present invention. In this technique, the inflow cannula is inserted into left ventricle 92, such as via mitral valve 110, for example by one of the following approaches:
transeptally via a right atrium 114 and an inferior vena cava 116 (e.g., using a transfemoral venous approach), such as shown,
via LAA 100, such as described hereinabove with reference to FIG. 5C, or
through the left ventricle's apex 94, such as described hereinabove with reference to FIG. 5A.

Alternatively, the inflow cannula is inserted into left atrium 108 via LAA 100, such as described hereinabove with reference to FIG. 5B.

For some applications, such as shown in FIG. 5D, the outflow cannula is coupled to a left subclavian artery 118 at first site 36A, typically by anastomosis. Alternatively, for some applications, the outflow cannula is coupled to descending aorta 104 at first site 36A, typically by anastomosis, such as described hereinabove with reference to FIG. 5C.

Alternatively, for some applications (configurations not shown), the outflow cannula is coupled to ascending aorta 96, typically by anastomosis, and/or the inflow cannula is inserted into left ventricle 92 through a wall of ascending aorta 96 and an aortic valve 97.

FIG. 5E shows another coupling technique, in accordance with respective applications of the present invention. In this technique, the inflow cannula is inserted into left atrium 108 via a venous approach, for example, transeptally via right atrium 114, such as shown. For example, the inflow cannula may be inserted into right atrium 114 via a superior vena cava 117, e.g., via a left subclavian vein 119, such as shown (such that inflow end opening 106 of the inflow cannula is within left atrium 108). Alternatively, the inflow cannula is further advanced and inserted into left ventricle 92 via mitral valve 110, such that inflow end opening 106 of the inflow cannula is within left ventricle 92, such as described hereinabove with reference to FIGS. 5C and 5D, mutatis mutandis. This technique for inserting the inflow cannula is similar in some respects to conventional techniques used for inserting pacemaker leads. Optionally, a pacemaker lead is coupled to the inflow cannula, thereby enabling the simultaneous placement of the inflow cannula and the pacemaker lead.

For some applications, such as shown in FIG. 5E, the outflow cannula is coupled to left subclavian artery 118 at first site 36A, typically by anastomosis. Alternatively, for some applications, the outflow cannula is coupled, typically by anastomosis, at first site 36A to another artery of the systemic circulation, such as, for example, (a) a left axillary artery, (b) descending aorta 104, such as described hereinabove with reference to FIG. 5C, or (c) ascending aorta 96.

Because the coupling technique described with reference to FIG. 5E is minimally invasive, it may be appropriate even for patients at early stages of heart failure, before more invasive procedures are appropriate.

For some applications, pulsatile-flow pump 24 is configured to pump 1-3 liters per minutes (lpm), e.g., 1.5-2.5 lpm, e.g., 2 lpm.

Reference is made to FIGS. 1-5E. In some applications of the present invention, LVAD system 10 is configured to operate in a counterpulsation mode, in which pump 24 is activated to pump blood downstream during diastole, rather than during systole in the normal operating mode described hereinabove. For example, in the configuration described hereinabove with reference to FIGS. 3A-B, control circuitry 56 is configured to activate tubular linear motor 44 to provide pulsatile flow synchronized with the cardiac cycles by, during each of the cardiac cycles: (a) activating stator 48, during at least a portion of diastole, to move magnetic piston 40 downstream during a first period of time having a first duration, and (b) activating stator 48, during at least a portion of systole, to move magnetic piston 40 upstream during a second period of time having a second duration. For some applications, the second duration is less than the first duration. The other techniques of this configuration described above may optionally be implemented, by swapping systole and diastole.

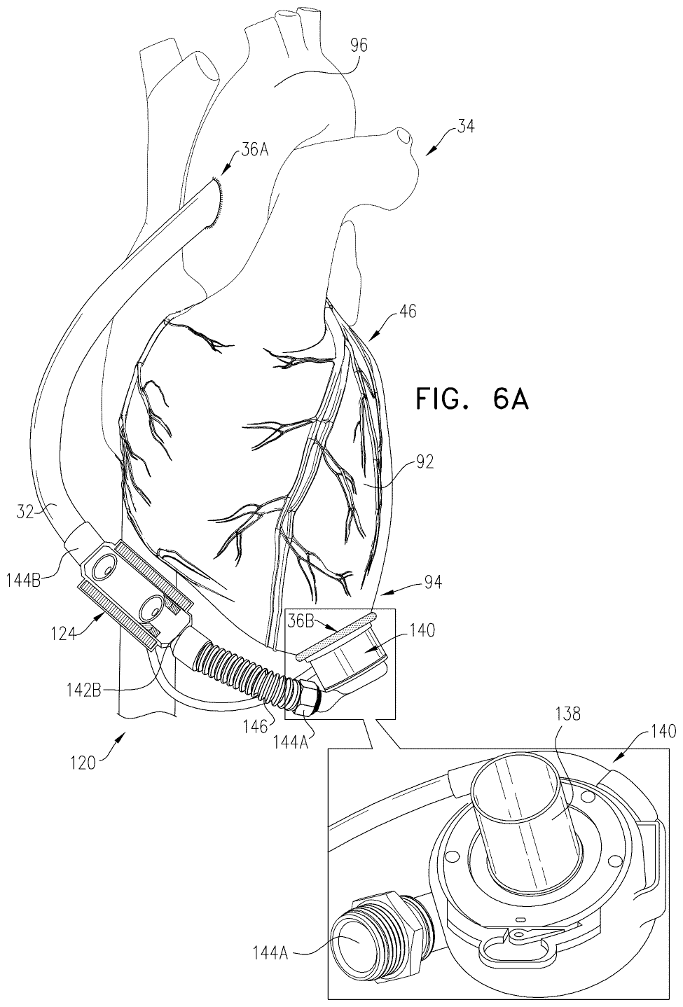

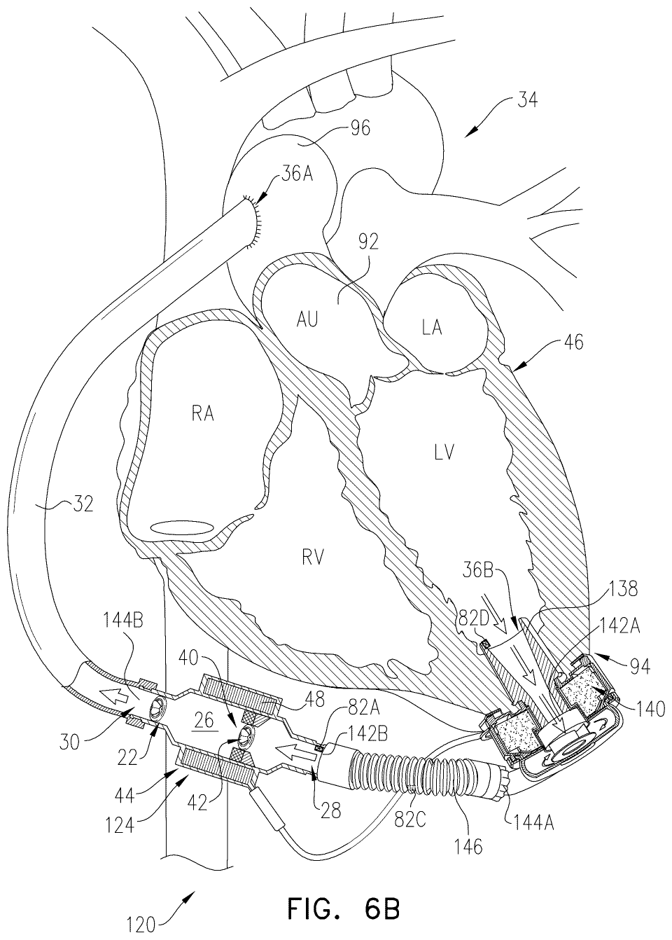

Reference is now made to FIGS. 6A-B, which are schematic illustrations of a left ventricular assist device (LVAD) 120 applied to a patient, in accordance with an application of the present invention. LVAD 120 is typically a component of an LVAD system, such as LVAD system 10, described hereinabove with reference to reference to FIG. 1. The LVAD system comprising LVAD 120 may implement any of the features of LVAD system 10 described hereinabove, mutatis mutandis, and LVAD 120 may implement any of the features of LVAD 20 described hereinabove, mutatis mutandis; for example, LVAD 120 may or may not comprise spring 50, and the control circuitry may or may not be configured to activate stator 48 to move magnetic piston 40 downstream at a velocity set based on a target stroke volume and a target stroke duration, during at least a portion of systole of each of the cardiac cycles.

LVAD 120 comprises:
outflow cannula 32, which is couplable in fluid communication with circulatory system 34 of the patient at first site 36A;
an inflow cannula 138, which is couplable in fluid communication with circulatory system 34 at a second site 36B upstream of first site 36A;
a continuous-flow pump 140, which comprises (1) a first inlet 142A in fluid communication with inflow cannula 138, and (2) a first outlet 144A; and
a pulsatile-flow pump 124, which comprises (1) a second inlet 142B and (2) a second outlet 144B, which is in fluid communication with outflow cannula 32.

Second inlet 142B of pulsatile-flow pump 124 is in fluid communication with first outlet 144A of continuous-flow pump 140, and thus with inflow cannula 138 via continuous-flow pump 140. Inflow cannula 138 is therefore arranged to allow downstream blood flow from inflow cannula 138 to second inlet 142B. Pulsatile-flow pump 124 may implement any of the features of pump 24, described hereinabove, mutatis mutandis, or may comprise a different kind of pulsatile-flow positive displacement pump known in the art.

The LVAD system comprising LVAD 120 further comprises control circuitry, which may implement any of the features of control circuitry 56, described hereinabove, mutatis mutandis. (The control circuitry is not shown in FIGS. 6A-B, but may be similar to circuitry 56 shown in FIG. 1 for LVAD system 10.) The control circuitry is typically configured to:
activate continuous-flow pump 140 to provide flow without synchronization with cardiac cycles of a heart of the patient, and
activate pulsatile-flow pump 124 to provide pulsatile flow synchronized with the cardiac cycles.

Alternatively, the control circuitry is configured to activate pulsatile-flow pump 124 to provide the pulsatile flow without synchronization with the cardiac cycles of the heart, such as at a constant or adjustable pulsation rate, e.g., 1 pulse per minute. This mode of operation may be appropriate, for example, for a very weak heart.

For some applications, the LVAD system comprising LVAD 120 further comprises cardiac sensor 52, which is configured to sense one or more features of the cardiac cycles, such as described hereinabove. The control circuitry is coupled to cardiac sensor 52.

For some applications, the control circuitry is configured to activate pulsatile-flow pump 124 to provide pulsatile flow synchronized with the cardiac cycles by, during each of the cardiac cycles, activating pulsatile-flow pump 124 to pump blood downstream during a portion of systole of the cardiac cycle, and not to pump blood downstream during any portion of diastole of the cardiac cycle, such as described hereinabove regarding control circuitry 56.

For some applications, the LVAD system comprising LVAD 120 further comprises stationary one-way outflow valve 22, which is arranged to allow downstream blood flow from second outlet 144B of pulsatile-flow pump 124 to outflow cannula 32, and to inhibit upstream blood flow from outflow cannula 32 to second outlet 144B.

For some applications, the LVAD system comprising LVAD 120 further comprises a tube 146 which couples second inlet 142B of pulsatile-flow pump 124 in the fluid communication with first outlet 144A of continuous-flow pump 140.

For some applications, pulsatile-flow pump 124:
is shaped so as to define pump chamber 26 having (a) upstream inflow end 28 in fluid communication with second inlet 142B, and (b) downstream outflow end 30 in fluid communication with second outlet 144B, and
comprises tubular linear motor 44, which comprises (a) magnetic piston 40, which comprises reciprocating one-way valve 42 configured to allow downstream blood flow and inhibit upstream blood flow; and (b) stator 48, which is configured to magnetically drive magnetic piston 40 with reciprocating motion, so as to pump blood downstream during downstream motion of magnetic piston 40 while reciprocating one-way valve 42 is closed.

For some of these applications, the control circuitry is configured to activate tubular linear motor 44 to provide the pulsatile flow synchronized with the cardiac cycles by, during each of the cardiac cycles:
activating stator 48 of tubular linear motor 44 to move magnetic piston 40 downstream during a portion of systole of the cardiac cycle, and
activating stator 48 of tubular linear motor 44 to move magnetic piston 40 upstream during a portion of diastole of the cardiac cycle.

For some applications, such as shown in FIG. 4 for LVAD system 10, the LVAD system comprising LVAD 120 further comprises stationary one-way inflow valve 72, which is arranged to allow downstream blood flow into pump chamber 26 of pulsatile-flow pump 124, and to inhibit upstream blood flow from pump chamber 26.

Continuous-flow pump 140 may comprise any type of continuous-flow pump known in the LVAD art. For example, continuous-flow pump 140 may comprise a magnetically-levitated centrifugal pump. Optionally, continuous-flow pump 140 comprises all or a portion of a commercially-available continuous-flow LVAD, such as, for example, the HeartMate 3 LVAD (St. Jude Medical, St. Paul, MN, USA), or HeartWare™ HVAD™ (Medtronic, Minneapolis, MN, USA).

For some applications, first site 36A is one of the first sites 36A described hereinabove with reference to FIG. 1 and/or FIGS. 1A-D.

For some applications, second site 36B is apex 94 of left ventricle 92 of heart 46, such as shown in FIGS. 6A-B, or one of the second sites 36B shown in FIGS. 1, 5B, or 5C.

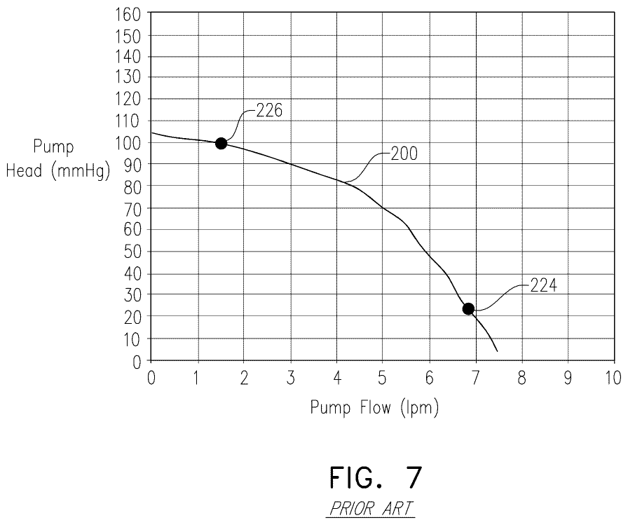

Reference is now made to FIG. 7, which is an illustrative pump curve 200 for a continuous-flow magnetically levitated centrifugal pump of a conventional LVAD, as known in the prior art. By way of example, pump curve 200 may be representative of a pump speed of 5,500 rpm. As indicated by pump curve 200, the higher the pump head (delta pressure across pump), the lower the pump flow.

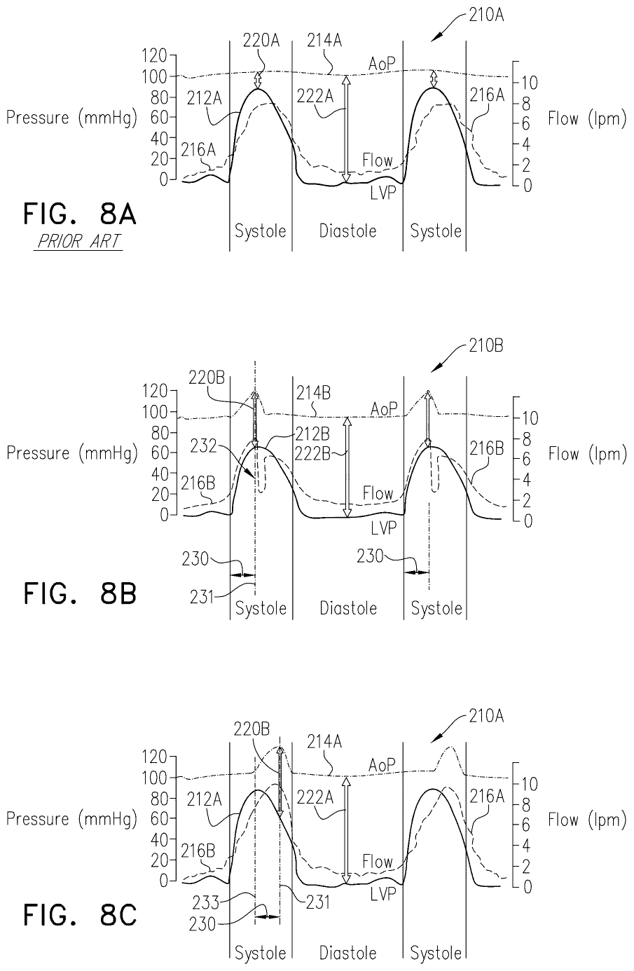

Reference is further made to FIG. 8A, which is an illustrative graph 210A showing blood pressure and blood flow rate for a continuous-flow magnetically levitated centrifugal pump of a conventional LVAD, as known in the prior art. Graph 210A includes:
a left-ventricular-pressure (LVP) curve 212A, measured in mmHg,
an aortic pressure (AoP) curve 214A, also measured in mmHg, and
a flow rate curve 216A, indicative of blood flow through the LVAD pump, measured in liters per minutes (lpm).

In a continuous-flow LVAD pump, for any given pump speed, the flow rate is driven largely by the delta pressure across the pump, i.e., the difference between AoP and LVP, as indicated by pump curve 200 of FIG. 7. As a result:
during systole, as LVP increases substantially, while AoP increases only slightly, the delta pressure is small, resulting in high flow, as indicated by a systolic delta arrow 220A in FIG. 8A and a systolic point 224 in FIG. 7; and
during diastole, as LVP decreases substantially, while AoP decreases only slightly, the delta pressure is large, resulting in low flow, as indicated by a diastolic delta arrow 222A in FIG. 8A a diastolic point 226 in FIG. 7.

As can be seen in AoP curve 214A, although conventional continuous-flow LVAD pumps produce some aortic pulsatility, the pulsatility is minimal. Because of this low pulsatility, continuous-flow LVADs are associated with altered arterial baroreceptors, because reduced pulsatility leads to increased sympathetic activation and peripheral vascular resistance.

Reference is now made to FIGS. 8B and 8C, which are illustrative graphs 210B showing blood pressure and blood flow rate for LVAD 120, in accordance with respective applications of the present invention. Graphs 210B include:
a left-ventricular-pressure (LVP) curve 212B, measured in mmHg,
an aortic pressure (AoP) curve 214B, also measured in mmHg, and
a flow rate curve 216B, indicative of blood flow through LVAD 120 (through both continuous-flow pump 140 and pulsatile-flow pump 124, in series), measured in liters per minute (lpm).

Graphs 210B reflect the combined effect of pulsatile-flow pump 124 and continuous-flow pump 140, as follows.

During a portion 230 of systole in which pulsatile-flow pump 124 pumps blood (as magnetic piston 40 moves downstream), blood flow through LVAD 120, as reflected by flow rate curve 216B, is entirely provided by pulsatile-flow pump 124 (even though continuous-flow pump 140 continues to pump constantly). (The flow rate during portion 230 of systole is thus not dependent on the delta pressure across the pump, unlike in conventional LVAD pumps, as described hereinabove with reference to FIG. 7.) Thus, during portion 230 of systole, the pressure increases substantially, as shown in FIGS. 8B-C, with a greater pressure increase than the pressure increase during the same portion of systole caused by a conventional continuous-flow LVAD pump, as shown in FIG. 8A. This increase in pressure results in a substantial increase in AoP during portion 230 of systole and for some time thereafter, as shown in FIGS. 8B-C. This substantial increase in AoP during systole provides substantial aortic pulsatility that mimics the natural aortic pulsatility in a healthy heart, avoiding the alteration of arterial baroreceptors, and resulting potential harmful effects on many organs, which may be caused by conventional continuous-flow LVAD pumps.

During the remainder of the cardiac cycle, typically including the remainder of systole and all of diastole, in which pulsatile-flow pump 124 does not pump blood (as magnetic piston 40 either moves upstream or is stationary), blood flow through LVAD 120, as reflected by flow rate curve 216B, is entirely driven by continuous-flow pump 140. During this portion of the cardiac cycle, reciprocating one-way valve 42 of magnetic piston 40 of pulsatile-flow pump 124 is open, as is stationary one-way outflow valve 22, if provided. As a result, left ventricle 92 comes into fluid communication with ascending aorta 96 via continuous-flow pump 140 and the open valve(s) of pulsatile-flow pump 124, and LVAD 120 thus behaves as a conventional continuous-flow pump, i.e., the flow rate is driven largely by the delta pressure across the pump, i.e., the difference between AoP and LVP, in accordance with pump curve 200 (for the given pump speed), as described hereinabove with reference to FIG. 7.

Typically, the portion 230 of systole in which pulsatile-flow pump 124 pumps blood has a duration of 200-400 milliseconds, e.g., 300-400 milliseconds, such as 300-350 milliseconds, and/or a duration of 20%-40% of a total duration of the cardiac cycle, e.g., 30%-40%, e.g., 30%-35% of the total duration of the cardiac cycle.

Reference is still made to FIGS. 8B-C. For some applications:
pulsatile-flow pump 124 is configured to pump 1-2 lpm, e.g., 1.25-1.75 lpm, e.g., 1.5 lpm, and/or
continuous-flow pump 140 is configured to pump 2.5-10 lpm, e.g., 3-6 lpm, e.g., 5 lpm.

Reference is made to FIG. 8B. For some applications, such as shown in FIG. 8B, the control circuitry is configured, during each of the cardiac cycles, to activate pulsatile-flow pump 124 to begin the portion 230 of systole at or near the beginning of systole, e.g., 0-50 milliseconds after the beginning of systole (at the Q deflection of the QRS complex).

Reference is still made to FIG. 8B. At a point 231 during systole at which tubular linear motor 44 of pulsatile-flow pump 124 completes its downstream stroke (upon conclusion of portion 230 of systole), the delta pressure is quite large, as indicated by a systolic delta arrow 220B in FIG. 8B. As a result of this high delta, continuous-flow pump 140 substantially cannot pump blood and the flow drops substantially (and typically steeply) at a portion 232 of flow rate curve 216B, until the pressure delta declines and continuous-flow pump 140 begins to pump blood, resulting in a fairly steep increase in flow after portion 232.

Reference is made to FIG. 8C. Alternatively, for some applications, such as shown in FIG. 8C, the control circuitry is configured, during each of the cardiac cycles, to activate pulsatile-flow pump 124 to begin the portion 230 of systole later during systole, i.e., at a later point 233 during systole. For example, the control circuitry may be configured to activate pulsatile-flow pump 124 to begin the portion 230 of systole at a pre-set or a calculated amount of time after the beginning of systole. For example, the control circuitry may be configured to activate pulsatile-flow pump 124 to begin the portion 230 of systole at a delay after the beginning of systole, the delay typically having:
a duration of 200-400 milliseconds, e.g., 300-400 milliseconds, such as 300-350 milliseconds, and/or
a duration equal to 20%-40% of a total duration of the cardiac cycle, e.g., 30%-40%, e.g., 30%-35% of the total duration of the cardiac cycle (e.g., as estimated based on previous recent cardiac cycles, as ascertained using cardiac sensor 52).

Alternatively, for some applications, the control circuitry is configured to activate pulsatile-flow pump 124 to begin the portion 230 of systole shortly before, at, or shortly after peak blood flow during systole. For example, the control circuitry may be configured to activate pulsatile-flow pump 124 to begin the portion 230 of systole between 50 milliseconds (e.g., 25 milliseconds) before and 50 milliseconds (e.g., 25 milliseconds) after peak blood flow during systole.

Further alternatively, for some applications, the control circuitry is configured to activate pulsatile-flow pump 124 to begin the portion 230 of systole upon detection (e.g., 0-25 milliseconds after detection) by the control circuitry of a beginning of a decline in pressure after a rise in pressure during systole. To this end, such as shown in FIG. 6B, the LVAD system typically comprises an upstream pressure sensor, which is disposed and configured to measure blood pressure of blood upstream of magnetic piston 40 of pulsatile-flow pump 124. For example, the blood pressure sensor may comprise:
upstream pressure sensor 82A, described hereinabove with reference to FIG. 2, which is disposed and configured to measure blood pressure of blood entering upstream inflow end 28 of pump chamber 26 of pulsatile-flow pump 124,
an upstream pressure sensor 82C, which is disposed along tube 146 (which couples second inlet 142B of pulsatile-flow pump 124 in the fluid communication with first outlet 144A of continuous-flow pump 140), and which is configured to measure blood pressure of blood entering upstream inflow end 28 of pump chamber 26 of pulsatile-flow pump 124, or
an upstream pressure sensor 82D, which is disposed upstream of continuous-flow pump 140, and configured to measure left-ventricular pressure (LVP); for example, upstream pressure sensor 82D may be coupled to inflow cannula 138. (Although all three pressure sensors 82A, 82C, and 82D are shown in FIG. 6B, in practice the LVAD system typically comprises only one of these sensors in configurations in which upstream blood pressure is measured.)

Even though upstream pressure sensors 82A and 82C, on the one hand, and upstream pressure sensor 82D, on the other hand, measure different pressures from each other, the above-mentioned decline in pressure occurs at substantially the same time in both pressures.

Reference is again made to FIG. 8C. As described above regarding the timing of activation of pulsatile-flow pump 124 described hereinabove with reference to FIG. 8B, at point 231 during systole at which tubular linear motor 44 of pulsatile-flow pump 124 completes its downstream stroke (upon conclusion of portion 230 of systole), the delta pressure is quite large, as indicated by systolic delta arrow 220B in FIG. 8B, as well as in FIG. 8C. As a result of this high delta, continuous-flow pump 140 substantially cannot pump blood and the flow drops substantially (and typically steeply) at portion 232 of flow rate curve 216B labeled in FIG. 8B, until the pressure delta declines and continuous-flow pump 140 begins to pump blood. By contrast, in the timing of pulsatile-flow pump 124 illustrated in FIG. 8C, the drop in flow coincides with the natural drop in flow toward the end of systole. As a result, the portion 232 of flow rate curve 216B shown in FIG. 8B is substantially subsumed by the natural drop, and the fairly steep increase in flow after portion 232 of FIG. 8B is absent in FIG. 8C.

The different beginning-point techniques may be implemented in combination. For example, the control circuitry is configured to activate pulsatile-flow pump 124 to begin the portion 230 of systole upon the detection by the LVAD system of the beginning of the decline in the pressure after the rise in the pressure during systole, provided that this decline occurs at the pre-set or the calculated amount of time after the beginning of systole, as described above.

As described above, LVAD 120 advantageously improves aortic pulsatility compared to conventional continuous-flow LVAD pumps. In addition, in LVAD 120, unlike conventional continuous-flow LVAD pumps, malfunction of the continuous-flow pump cannot result in death, because, even in the event of such malfunction, pulsatile-flow pump 124 continues to pump blood. Optionally, LVAD is configured to increase the blood flow provided by pulsatile-flow pump 124 in the event of failure of continuous-flow pump 140.

In addition, LVAD 120 may have several advantages compared to LVAD 20, described hereinabove with reference to FIGS. 1-5E. Pulsatile-flow pump 124 of LVAD 120 typically requires a smaller stroke volume than pulsatile-flow pump 24 of LVAD 20, because continuous-flow pump 140 provides a portion of the blood flow of each cardiac cycle, when pulsatile-flow pump 124 is not pumping, as described above. Therefore, pulsatile-flow pump 124 can be smaller than pulsatile-flow pump 24, and typically consumes less power. This smaller size may simplify and/or otherwise facilitate implantation of pulsatile-flow pump 124.

In addition, LVAD 120 can be implanted using the same conventional techniques used for implanting conventional continuous-flow LVAD pumps, because:
continuous-flow pump 140, including inflow cannula 138, may be implantable and attachable to apex 94 of left ventricle 92 at second site 36B using conventional LVAD implantation techniques, and
outflow cannula 32 may be coupled in fluid communication with circulatory system 34 at first site 36A using conventional LVAD implantation techniques.

Pulsatile-flow pump 124 of LVAD 120 is effectively disposed along the outflow cannula of the continuous-flow pump, such that the presence of the pulsatile-flow pump does not materially alter the conventional implantation procedure with which cardiac surgeons are familiar. Optionally, the outflow cannula of continuous-flow pump 140 is provided to the surgeon with pulsatile-flow pump 124 disposed along the cannula. Continuous-flow pump 140 may be provided to the surgeon disconnected from the cannula, and the surgeon may connect the cannula, including pulsatile-flow pump 124, to continuous-flow pump 140. Alternatively, pulsatile-flow pump 124 is pre-coupled in fluid communication to continuous-flow pump 140 during manufacture.

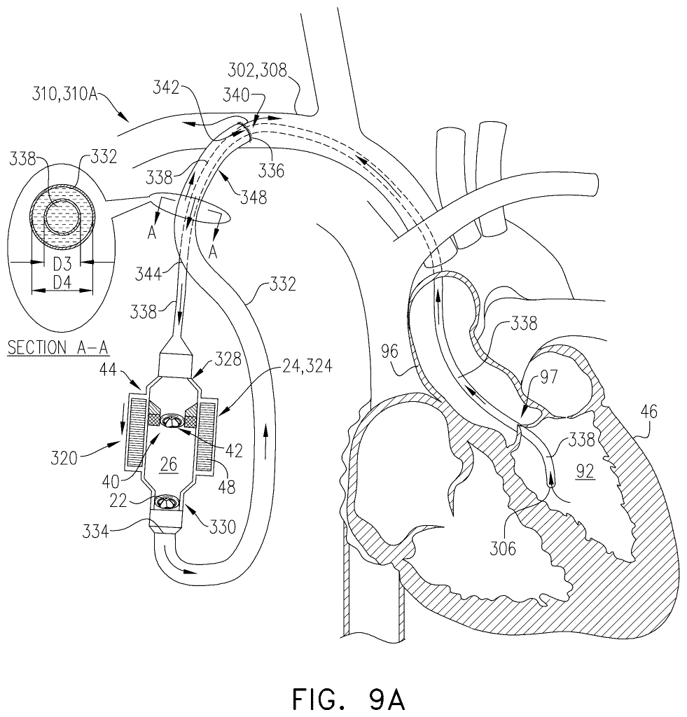

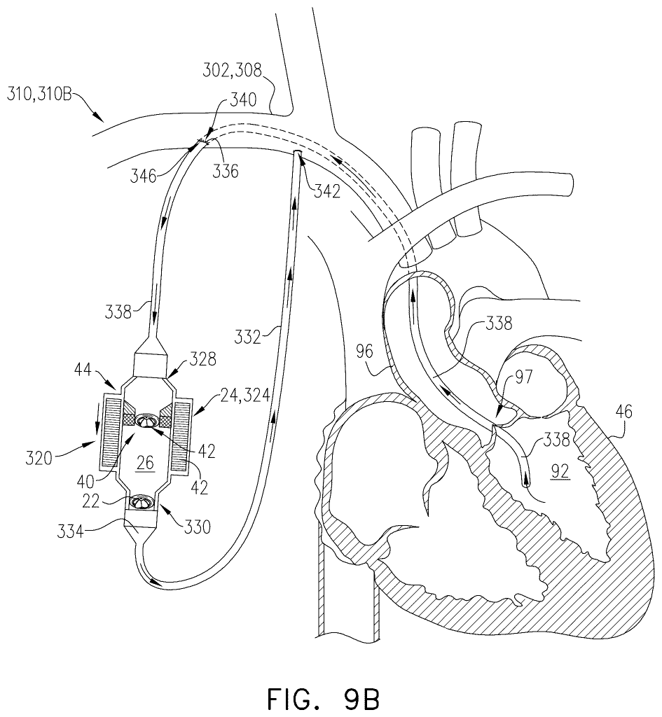

Reference is now made to FIGS. 9A and 9B, which are schematic cross-sectional illustrations of an LVAD system 310 applied to a patient, in accordance with respective applications of the present invention. LVAD system 310 may optionally implement any of the features of LVAD system 10, described hereinabove with reference to FIGS. 1-4E, mutatis mutandis, and like reference numerals refer to like parts.

LVAD system 310 comprises an implantable LVAD 320 for implantation in the patient. LVAD 320 comprises:
a pulsatile-flow pump 324, which comprises an upstream inlet 328 and a downstream outlet 330;
an inflow cannula 338, which is (1) configured to be inserted into left ventricle 92 of the patient via aortic valve 97 and ascending aorta 96, and (2) arranged in fluid communication with upstream inlet 328 of pulsatile-flow pump 324; and
an outflow cannula 332, which is (1) couplable to an artery 302 of the systemic circulation of the patient, and (2) arranged in fluid communication with downstream outlet 330 of pulsatile-flow pump 324. LVAD system 310 typically further comprises:
cardiac sensor 52 (shown by way of example in FIG. 1), configured to sense one or more features of a cardiac cycle of heart 46;
control circuitry 56 (shown by way of example in FIG. 1), which is coupled to cardiac sensor 52 and configured to activate pulsatile-flow pump 324 to provide pulsatile flow synchronized with the cardiac cycle in a counterpulsation mode, by pumping blood in a downstream direction out of downstream outlet 330 during at least a portion of ventricular diastole; optionally, control circuitry 56 may implement any of the features described hereinabove with reference to FIGS. 1-4E, mutatis mutandis; and
typically, power source 58 (shown by way of example in FIG. 1) (e.g., comprising one or more batteries) arranged to provide power to pulsatile-flow pump 324.

By drawing blood out of left ventricle 92 during diastole, LVAD system 310 may reduce the ventricular filling pressure and/or reduce atrial pressure, thereby preventing congestion; reduce ventricle wall stress; and/or elevate diastolic pressure. For some applications, LVAD system 310 further comprises stationary one-way outflow valve 22, which is arranged to allow downstream blood flow from downstream outlet 330 of pulsatile-flow pump 324 to outflow cannula 332, and to inhibit upstream blood flow from outflow cannula 332 to downstream outlet 330.

For some applications, pulsatile-flow pump 324 comprises a positive displacement (PD) pump.

For some of these applications, pulsatile-flow pump 324 comprises pump 24, described hereinabove with reference to FIGS. 1-4E, mutatis mutandis, and may implement any of the features of pump 24, mutatis mutandis. In these applications, pulsatile-flow pump 324 comprises:
pump chamber 26, which is shaped so as to define (a) upstream inlet 328 (corresponding to upstream inflow end 28, described hereinabove with reference to FIGS. 1-4E), and (b) downstream outlet 330 (corresponding to downstream outflow end 30, described hereinabove with reference to FIGS. 1-4E); and
tubular linear motor 44, which comprises (a) magnetic piston 40, which comprises reciprocating one-way valve 42 configured to allow downstream blood flow through reciprocating one-way valve 42, and inhibit upstream blood flow through reciprocating one-way valve 42; and (b) stator 48, which is configured to magnetically drive magnetic piston 40 with reciprocating motion, so as to pump blood in the downstream direction out of downstream outlet 330 during downstream motion of magnetic piston 40 while reciprocating one-way valve 42 is closed.

Typically, control circuitry 56 is configured to activate pulsatile-flow pump 324 to provide the pulsatile flow synchronized with the cardiac cycle in the counterpulsation mode by:
activating stator 48 to move magnetic piston 40 downstream during the at least a portion of the ventricular diastole, and
activating stator 48 to move magnetic piston 40 upstream during at least a portion of ventricular systole of the cardiac cycle (because reciprocating one-way valve 42 opens during upstream motion of magnetic piston 40, magnetic piston 40 does not pump blood in the upstream direction during upstream motion of magnetic piston 40).

For other applications, pulsatile-flow pump 324 comprises another type of pump known in the art.

Reference is still made to FIGS. 9A and 9B. For some applications, an upstream end opening 334 of outflow cannula 332 is arranged in the fluid communication with downstream outlet 330 of pulsatile-flow pump 324, and a downstream end 336 of outflow cannula 332 is configured to be anastomosed to artery 302 such that a downstream end opening 340 of outflow cannula 332 is in fluid communication with artery 302 via an arteriotomy 342.

Reference is still made to FIGS. 9A and 9B. In some applications of the present invention, a method for treating a patient is provided, which comprises:
implanting LVAD 320 of LVAD system 310 in the patient, by (a) inserting inflow cannula 338 of LVAD 320 into left ventricle 92 via aortic valve 97 and ascending aorta 96, (b) coupling outflow cannula 332 of LVAD 320 to artery 302 of the systemic circulation of the patient; and
activating control circuitry 56 of LVAD system 310 to activate pulsatile-flow pump 324 to provide pulsatile flow synchronized with a cardiac cycle of the patient in a counterpulsation mode, by pumping blood in a downstream direction out of downstream outlet 330 during at least a portion of ventricular diastole.

For some applications, artery 302 is selected from a right axillary artery and a right subclavian artery 308, and coupling outflow cannula 332 to artery 302 comprises coupling outflow cannula 332 to the selected artery. Alternatively, artery 302 is a different artery, such as an ascending aorta, an aortic arch, or a descending aorta.

For some applications, coupling outflow cannula 332 to artery 302 comprises anastomosing downstream end 336 of outflow cannula 332 to artery 302 such that downstream end opening 340 of outflow cannula 332 is in fluid communication with artery 302 via arteriotomy 342.

Reference is now made to FIG. 9A. In some applications of the present invention, LVAD system 310 comprises a LVAD system 310A. In this configuration, an outer cross-sectional area of inflow cannula 338 is less than an inner cross-sectional area of outflow cannula 332, the cross-sectional areas measured perpendicular to respective longitudinal axes of cannulas 332 and 338. (The outer cross-sectional area includes the thickness of the wall of inflow cannula 338, and is based on an outer diameter D3 of inflow cannula 338, while the inner cross-sectional area does not include the thickness of the wall of outflow cannula 332, and is based on an inner diameter D4 of outflow cannula 332.)

In this configuration, outflow cannula 332 is shaped so as to define a lateral opening 344. Inflow cannula 338 is arranged passing through (a) downstream end opening 340 of outflow cannula 332, (b) a longitudinal portion 348 of outflow cannula 332, and (c) lateral opening 344 of outflow cannula 332, so as to (i) form a liquid-tight seal between an outer surface of inflow cannula 338 and lateral opening 344, and (ii) allow blood flow within outflow cannula 332 outside and alongside inflow cannula 338. Inflow cannula 338 is configured to be positioned passing through arteriotomy 342.

For some applications, LVAD system 310A is configured such that inflow cannula 338 is slidable through lateral opening 344 of outflow cannula 332, optionally while maintaining the liquid-tight seal between the outer surface of inflow cannula 338 and lateral opening 344. For example, outflow cannula 332 may comprise an O-ring or other seal at the border of lateral opening 344. Inflow cannula 338 may be inserted through lateral opening 344 of outflow cannula 332 prior or during the implantation procedure. In configurations in which inflow cannula 338 is inserted through lateral opening 344 of outflow cannula 332 during the implantation procedure, the surgeon may optionally fix and/or seal inflow cannula 338 to lateral opening 344 of outflow cannula 332, such as by suturing or gluing; optionally, the liquid-tight seal between the outer surface of inflow cannula 338 and lateral opening 344 is only established after the surgeon fixes and/or seals inflow cannula 338 to lateral opening 344 of outflow cannula 332.

Optionally, inflow cannula 338 is initially not disposed within outflow cannula 332 and not coupled to pulsatile-flow pump. During the implantation procedure, the surgeon inserts inflow cannula 338 through downstream end 336 of outflow cannula 332 and out of lateral opening 344 of outflow cannula 332, and then couples inflow cannula 338 to the pulsatile-flow pump. Optionally, the surgeon inserts inflow cannula 338 through downstream end 336 of outflow cannula 332 after anastomosing downstream end 336 of outflow cannula 332 to artery 302. Alternatively, the surgeon may optionally implant the elements of LVAD system 310A in a different order while inflow cannula 338 is not coupled to the pulsatile-flow pump, and subsequently couple inflow cannula 338 to the pulsatile-flow pump.

For other applications, LVAD system 310A is configured such that inflow cannula 338 is fixed to lateral opening 344 of outflow cannula 332.

This arrangement allows the implantation of both outflow cannula 332 and inflow cannula 338 by making only a single arteriotomy 342 and performing only a single anastomosis, which may simplify the implantation procedure.

For some applications, a length of inflow cannula 338 outside outflow cannula 332, between (a) an inflow end opening 306 of inflow cannula 338 and (b) downstream end opening 340 of outflow cannula 332, is at least 20 cm, no more than 40 cm, and/or 20-40 cm.

For some applications, inserting inflow cannula 338 into left ventricle 92 comprises inserting inflow cannula 338 through arteriotomy 342 and into artery 302, and advancing inflow cannula 338 through artery 302, ascending aorta 96, and aortic valve 97, into left ventricle 92.

For some applications, (a) a volume of outflow cannula 332, outside inflow cannula 338, between downstream end opening 340 and lateral opening 344 of outflow cannula 332 is approximately equal to (e.g., 80%-120%, such as 90%-110% of) (b) a volume of inflow cannula 338 between downstream end opening 340 and lateral opening 344 of outflow cannula 332.

The outer diameter D3 of inflow cannula 338 is less than the inner diameter D4 of outflow cannula 332, such as at least 65%, no more than 75%, and/or 65%-75% of the inner diameter of outflow cannula 332. Thus, the outer cross-sectional area of inflow cannula 338 may be 65%-70% of the inner cross-sectional area of outflow cannula 332, such that the inner cross-sectional area of inflow cannula 338 may be approximately equal to the inner cross-sectional area of outflow cannula 332 less the outer cross-sectional area of inflow cannula 338, in order to provide approximately equal cross-sectional areas for blood flow in both directions.

For some applications, the outer diameter D3 of inflow cannula 338 is at least 8 mm, no more than 13 mm, and/or 8-13 mm.

For some applications, an outer diameter of outflow cannula 332 is at least 12 mm, no more than 18 mm, and/or 12-18 mm.

Reference is still made to FIG. 9A. In some applications of the present invention, a ventricular assist device (VAD) system for treating a patient is provided. The VAD system may implement any of the features of LVAD system 310, described hereinabove with reference to FIGS. 9A and 9B, and/or LVAD system 310A, described hereinabove with reference to FIG. 9A. The VAD system comprises:
an implantable VAD for implantation in the patient, the VAD comprising (a) a pump, which comprises an upstream inlet and a downstream outlet; (b) an inflow cannula, which is (1) arranged in fluid communication with the upstream inlet of the pump, and (2) configured to be coupled to a circulatory system of the patient in fluid communication with a first site of the circulatory system; and (c) an outflow cannula, which has (1) an upstream end opening arranged in fluid communication with the downstream outlet of the pump, and (2) a downstream end configured to be anastomosed to an artery of the patient at a second site, such that a downstream end opening of the outflow cannula is in fluid communication with the artery via an arteriotomy; and
control circuitry, which is coupled to the cardiac sensor and configured to activate the pump to provide blood flow.

An outer cross-sectional area of the inflow cannula is less than an inner cross-sectional area of the outflow cannula, the cross-sectional areas measured perpendicular to respective longitudinal axes of the cannulas. The outflow cannula is shaped so as to define a lateral opening. The inflow cannula is arranged passing through (a) the downstream end opening of the outflow cannula, (b) a longitudinal portion of the outflow cannula, and (c) the lateral opening of the outflow cannula, so as to (i) form a liquid-tight seal between an outer surface of the inflow cannula and the lateral opening, and (ii) allow blood flow within the outflow cannula outside and alongside the inflow cannula. The inflow cannula is configured to be positioned passing through the arteriotomy.

For some applications, the pump comprises a pulsatile-flow pump. Alternatively, for some applications, the pump comprises a continuous-flow pump. The implantable VAD further comprises a cardiac sensor, configured to sense one or more features of a plurality of cardiac cycles of a heart of the patient. The control circuity is configured to activate the pulsatile-flow pump to provide pulsatile flow synchronized with the cardiac cycles.

Reference is still made to FIG. 9A. In some applications of the present invention, a method for treating a patient is provided. The method may implement any of the techniques described hereinabove with reference to FIGS. 9A and 9B and/or with reference to FIG. 9A.

The method comprises implanting a ventricular assist device (VAD) of a VAD system in the patient, by:
making an arteriotomy through an artery of the patient at a second site of a circulatory system of the patient;
passing an inflow cannula of the VAD through the arteriotomy and coupling the inflow cannula to the circulatory system in fluid communication with a first site of the circulatory system; and
anastomosing a downstream end of an outflow cannula of the VAD to the artery of the patient at the second site, such that a downstream end opening of the outflow cannula is in fluid communication with the artery via the arteriotomy.

The VAD includes a pump, which includes (a) an upstream inlet in fluid communication with the inflow cannula, and (b) a downstream outlet in fluid communication with an upstream end opening of the outflow cannula.

The method further comprises activating control circuitry of the VAD system to activate the pump to provide blood flow. An outer cross-sectional area of the inflow cannula is less than an inner cross-sectional area of the outflow cannula, the cross-sectional areas measured perpendicular to respective longitudinal axes of the cannulas. The inflow cannula is arranged passing through (a) the downstream end opening of the outflow cannula, (b) a longitudinal portion of the outflow cannula, and (c) the lateral opening of the outflow cannula, so as to (i) form a liquid-tight seal between an outer surface of the inflow cannula and the lateral opening, and (ii) allow blood flow within the outflow cannula outside and alongside the inflow cannula.

For some applications, the first site of the circulatory system is a left ventricle of the patient, and coupling the inflow cannula to the circulatory system in fluid communication with the first site of the circulatory system comprises inserting the inflow cannula through the arteriotomy and into the artery, and advancing the inflow cannula through the artery, an ascending aorta, and an aortic valve, into the left ventricle.

For some applications, implanting the VAD comprises implanting the VAD such that the first and the second sites are arterial first and second sites of the circulatory system, such that activating control circuitry causes arterial blood to flow within both the inflow cannula and outflow cannula.

For some applications, implanting the VAD comprises implanting the VAD such that the first and the second sites are venous first and second sites of the circulatory system, such that activating control circuitry causes venous blood to flow within both the inflow cannula and outflow cannula.

Reference is now made to FIG. 9B. In some applications of the present invention, LVAD system 310 comprises an LVAD system 310B. In this configuration, unlike the configuration of LVAD system 310A described hereinabove with reference to FIG. 9A, inflow cannula 338 is not arranged passing through a longitudinal portion of outflow cannula 332. Instead, arteriotomy 342 is a first arteriotomy 342, and inflow cannula 338 is inserted into artery 302 via a second arteriotomy 346 and anastomosed to the wall of the artery around the second arteriotomy.

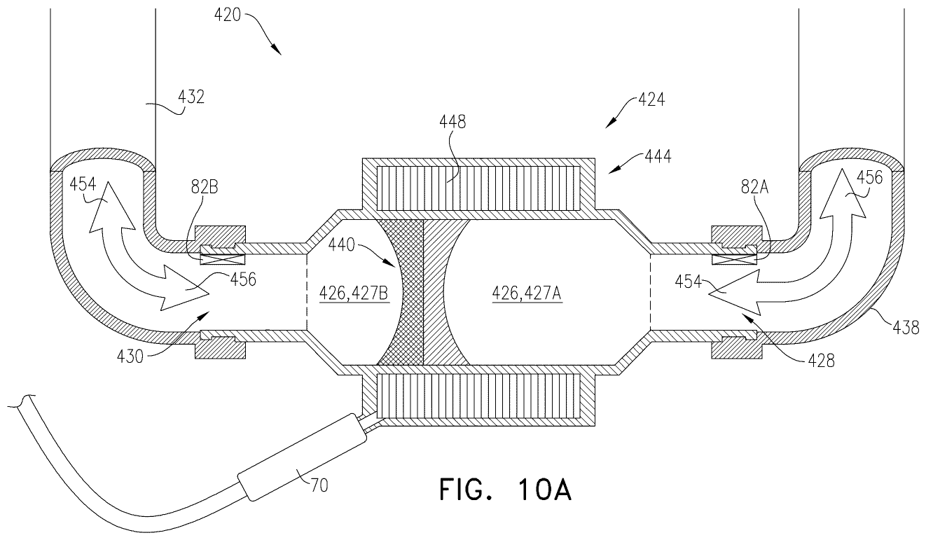

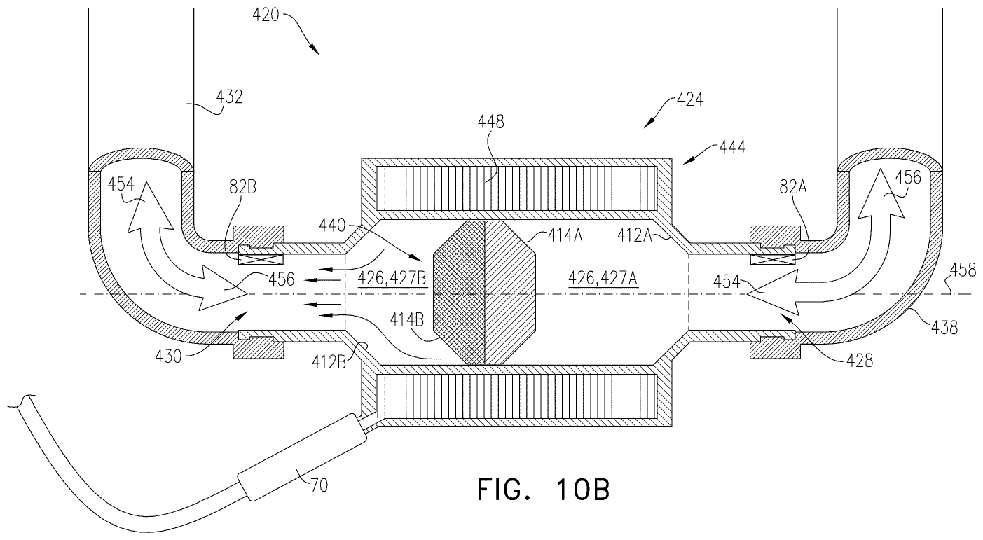

Reference is now made to FIGS. 10A and 10B, which are schematic cross-sectional illustrations of a right ventricular assist device (RVAD) system 410, in accordance with respective applications of the present invention. RVAD system 410 may optionally implement any of the features of LVAD system 10, described hereinabove with reference to FIGS. 1-4E, mutatis mutandis, and like reference numerals refer to like parts.

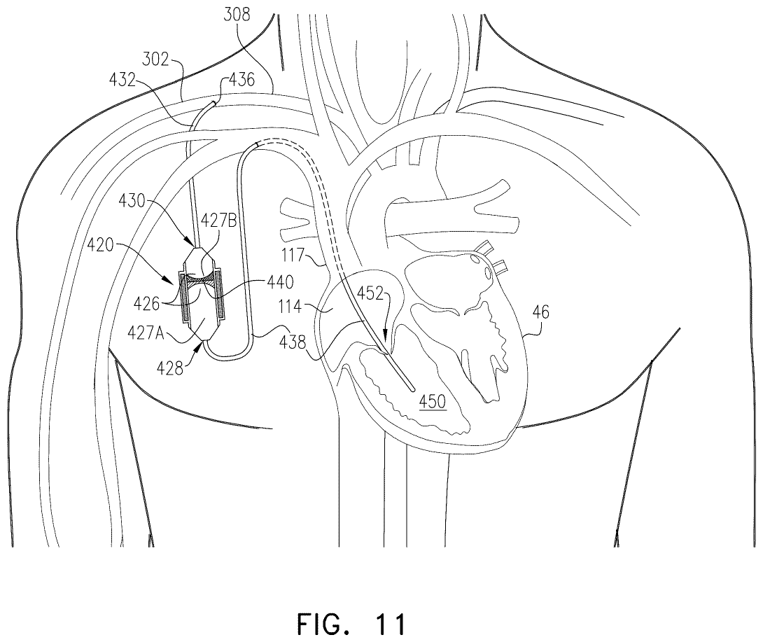

Reference is further made to FIG. 11, which is a schematic cross-sectional illustration of RVAD system 410 applied to a patient, in accordance with an application of the present invention.

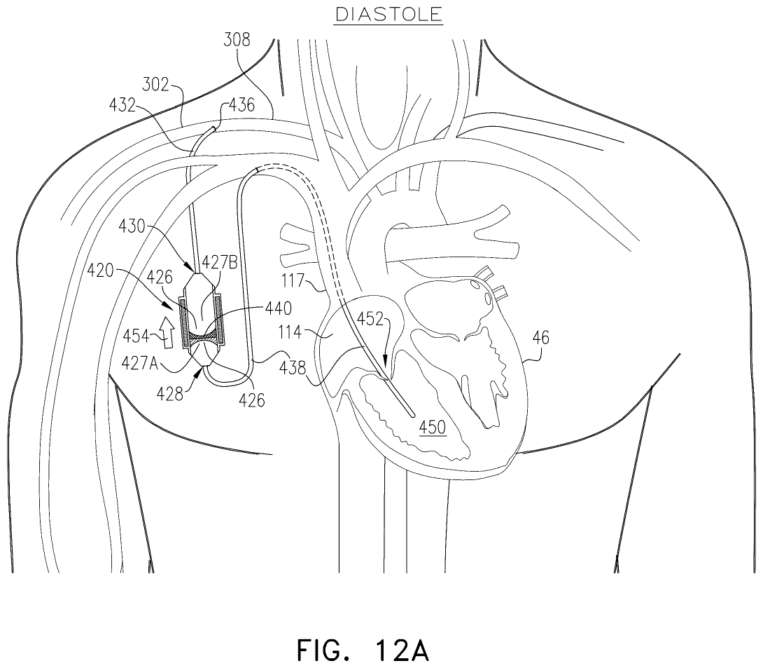

Reference is still further made to FIGS. 12A-B, which are schematic cross-sectional illustrations of operation of RVAD system 410 when applied to a patient, in accordance with an application of the present invention.

RVAD system 410 comprises an implantable RVAD 420 for implantation in the patient. RVAD 420 comprises:
a pulsatile-flow pump 424, which is shaped so as to define a pump chamber 426, which includes a first sub-chamber 427A having a proximal opening 428, and a second sub-chamber 427B having a distal opening 430 (the vertical dashed lines demarcate one end of each of first sub-chamber 427A and second sub-chamber 427B, and thus both ends of pump chamber 426);
a proximal cannula 438, which is (1) configured to be inserted into a right ventricle 450 of the patient, and (2) arranged in fluid communication with proximal opening 428 of first sub-chamber 427A of pump chamber 426; and
a distal cannula 432, which is (1) couplable to an artery 302 of the systemic circulation of the patient, and (2) arranged in fluid communication with distal opening 430 of second sub-chamber 427B of pump chamber 426.

First and second sub-chambers 427A and 427B are in fluid isolation from each other, such that proximal opening 428 and distal opening 430 are in fluid isolation from each other (via RVAD 420; they may be in indirect fluid communication with each other via the cardiac circulation). First and second sub-chambers 427A and 427B have first and second variable volumes, respectively, the sum of which is the volume of pump chamber 426 (which does not include the volume occupied by magnetic piston 440, if provided, as described hereinbelow). During operation of pulsatile-flow pump 424, the first volume increases as the second volume decreases, and vice versa.

RVAD system 410 typically further comprises:
cardiac sensor 52 (shown by way of example in FIG. 1), configured to sense one or more features of a cardiac cycle of heart 46;
control circuitry 56 (shown by way of example in FIG. 1), which is configured to activate pulsatile-flow pump 424 to provide pulsatile flow synchronized with the cardiac cycle in a counterpulsation mode, by:
   during at least a portion of ventricular diastole, pumping blood in a distal direction 454 out of distal opening 430 of second sub-chamber 427B of pump chamber 426 and drawing blood in distal direction 454 into proximal opening 428 of first sub-chamber 427A of pump chamber 426, such as shown in FIG. 12A, and
   during at least a portion of ventricular systole, pumping blood in a proximal direction 456 out of proximal opening 428 of first sub-chamber 427A of pump chamber 426 and drawing blood in proximal direction 456 into distal opening 430 of second sub-chamber 427B of pump chamber 426, such as shown in FIG. 12B; and
typically, power source 58 (shown by way of example in FIG. 1) (e.g., comprising one or more batteries) arranged to provide power to pulsatile-flow pump 424.

Thus, during diastole, pulsatile-flow pump 424 draws from right ventricle 450, thereby reducing venous pressure and pushing blood to the arterial systemic circulation. During systole, pulsatile-flow pump 424 draws blood from the arterial systemic circulation and reduces the pressure on left ventricle 92.

Typically, RVAD 420 does not comprise valves at either proximal opening 428 of first sub-chamber 427A or distal opening 430 of second sub-chamber 427B of pump chamber 426, in order to allow bidirectional flow through pulsatile-flow pump 424.

For some applications, proximal cannula 438 has an outer diameter of at least 8 mm, no more than 13 mm, and/or 8-13 mm, and/or a length of at least 15 cm, no more than 40 cm, and/or 15-40 cm.

Typically, RVAD system 410 is configured to contain blood only within pump chamber 426 and proximal and distal cannulas 438 and 432.

For some applications, proximal cannula 438 is configured to be inserted into right ventricle 450 via a vena cava (such as superior vena cava 117), right atrium 114, and a tricuspid valve 452, and as shown in FIGS. 11 and 12A-B.

For some applications, an end 436 of distal cannula 432 is configured to be anastomosed to artery 302.

As described above, first and second sub-chambers 427A and 427B have first and second variable volumes, respectively, the sum of which is the volume of pump chamber 426. For some applications, the volume of pump chamber 426 (a) is greater than (e.g., at least 20% greater than) a volume of RVAD 420 distal to second sub-chamber 427B and (b) is greater than (e.g., at least 20% greater than) a volume of RVAD 420 proximal to first sub-chamber 427A. As a result, during each cardiac cycle, a portion of the blood contained within both first sub-chamber 427A and second sub-chamber 427B is replaced with new blood drawn in from the blood circulation. This constant replacement of blood reduces the risk of coagulation within first sub-chamber 427A and second sub-chamber 427B that might occur if the same blood remained within the sub-chambers for multiple cardiac cycles.

As used herein, the volume of RVAD 420 distal to second sub-chamber 427B is the sum of the volume of distal cannula 432 and the volume of any portions of pulsatile-flow pump 424 distal to second sub-chamber 427B, such as a portion of pulsatile-flow pump 424 near distal opening 430.

As used herein, the volume of RVAD 420 proximal to first sub-chamber 427A is the sum of the volume of proximal cannula 438 and the volume of any portions of pulsatile-flow pump 424 proximal to first sub-chamber 427A, such as a portion of pulsatile-flow pump 424 near proximal opening 428.

Optionally, the volume of pump chamber 426 is at least 50% greater than and/or no more than 100% greater than the volume of RVAD 420 distal to second sub-chamber 427B.

Optionally, the volume of pump chamber 426 is at least 50% greater than and/or no more than 100% greater than the volume of RVAD 420 proximal to first sub-chamber 427A.

For some applications, pulsatile-flow pump 424 comprises a positive displacement (PD) pump.

For some of these applications, pulsatile-flow pump 424 comprises a tubular linear motor 444, which comprises:
a magnetic piston 440 slidably disposed within pump chamber 426, fluidly isolating first and second sub-chambers 427A and 427B from each other; the longitudinal location of magnetic piston 440 within pump chamber 426 sets the relative volumes of first and second sub-chambers 427A and 427B; and
a stator 448, which is configured to magnetically drive magnetic piston 440 with reciprocating motion, so as to pump blood in distal direction 454 during distal motion of magnetic piston 440, and in proximal direction 456 during proximal motion of magnetic piston 440.

The use of magnetic piston 440 allows for a pump that is entirely closed, without the need for any elements external to the pump to be connected to the piston.

Typically, control circuitry 56 is configured to activate pulsatile-flow pump 424 to provide the pulsatile flow synchronized with the cardiac cycle in the counterpulsation mode, by:
during the at least a portion of ventricular diastole, activating stator 448 to pump blood in distal direction 454 out of distal opening 430 of second sub-chamber 427B of pump chamber 426 and to draw blood in distal direction 454 into proximal opening 428 of first sub-chamber 427A of pump chamber 426, by moving magnetic piston 440 in distal direction 454, such as shown in FIG. 12A, and
during the at least a portion of ventricular systole, activating stator 448 to pump blood in proximal direction 456 out of proximal opening 428 of first sub-chamber 427A of pump chamber 426 and to draw blood in proximal direction 456 into distal opening 430 of second sub-chamber 427B of pump chamber 426, by moving magnetic piston 440 in proximal direction 456, such as shown in FIG. 12B.

Unlike magnetic piston 40, described hereinabove with reference to FIGS. 1-9B, magnetic piston 440 does not comprise a valve, in order to enable bidirectional pumping by fluidly isolating first and second sub-chambers 427A and 427B from each other.

Reference is made to FIG. 10B (it is noted that the figures, including FIG. 10B, are not drawn to scale, and that the relative sizes of elements are illustrative only). In this configuration, a greatest cross-sectional area of pump chamber 426 is (a) greater than a cross-sectional area of proximal opening 428 and (b) greater than a cross-sectional area of distal opening 430, all of the cross-sectional areas measured perpendicular to a central longitudinal axis 458 of pump chamber 426. A cross-sectional area of magnetic piston 440 equals or is slightly less than (e.g., is 98%-100% of) the greatest cross-sectional area of pump chamber 426. A proximal end wall 412A of first sub-chamber 427A of pump chamber 426 and a distal end wall 412B of second sub-chamber 427B of pump chamber 426 are frustoconical (as shown) or curved (configuration not shown). Peripheral portions of proximal and distal longitudinal ends 414A and 414B of magnetic piston 440 have respective shapes that correspond to the shapes of proximal and distal end walls 412A and 412B, respectively, such that ends 414A and 414B of magnetic piston 440 come in snug contact with proximal and distal end walls 412A and 412B and the proximal and distal ends of each stroke of magnetic piston 440. (In configurations in which proximal and distal end walls 412A and 412B are frustoconical, the peripheral portions of proximal and distal longitudinal ends 414A and 414B of magnetic piston 440 are also frustoconical, and have the same cone angles as proximal and distal end walls 412A and 412B.)

As a result, pump chamber 426 does not include substantial areas at the ends of the pump chamber in which blood remains stagnant during pumping, thereby reducing the risk of coagulation. By contrast, if, for example, proximal and distal end walls 412A and 412B of pump chamber 426 instead were shaped as a flat disc perpendicular to central longitudinal axis 458 of pump chamber 426, and ends 414A and 414B of magnetic piston 440 defined flat surfaces perpendicular to central longitudinal axis 458, more blood would pool near the ends of the pump chamber, which might increase the risk of coagulation. (Pulsatile-flow pump 424 is typically configured such that ends 414A and 414B of magnetic piston 440 do not reach and touch proximal and distal end walls 412A and 412B of pump chamber 426, respectively, and the ends of the stroke, to avoid banging on the end walls during each cardiac cycle.)

In addition, tubular linear motor 444 expels substantially all of the blood from each of first and second sub-chambers 427A and 427B during the respective proximal and distal movements of the magnetic piston during each cardiac cycle, because first and second sub-chambers 427A and 427B do not define any areas from which blood is not expelled each cardiac cycle. By contrast, providing areas from which blood is not expelled would increase the risk of coagulation.

Reference is still made to FIGS. 10A-B, 11, and 12A-B. In some applications of the present invention, a method for treating a patient is provided, which comprises:
implanting RVAD 420 of RVAD system 410 in the patient, by (a) inserting proximal cannula 438 of RVAD 420 into right ventricle 450 of heart 46, and coupling distal cannula 432 of RVAD 420 to an artery 302 of the systemic circulation of the patient; and
activating control circuitry 56 of RVAD system 410 to activate pulsatile-flow pump 424 to provide pulsatile flow synchronized with a cardiac cycle in a counterpulsation mode, by (a) during at least a portion of ventricular diastole, pumping blood in distal direction 454 out of distal opening 430 of second sub-chamber 427B of pump chamber 426 and drawing blood in distal direction 454 into proximal opening 428 of first sub-chamber 427A of pump chamber 426, such as shown in FIG. 12A, and (b) during at least a portion of ventricular systole, pumping blood in proximal direction 456 out of proximal opening 428 of first sub-chamber 427A of pump chamber 426 and drawing blood in proximal direction 456 into distal opening 430 of second sub-chamber 427B of pump chamber 426, such as shown in FIG. 12B.

For some applications, artery 302 is selected from a right axillary artery and right subclavian artery 308, and coupling distal cannula 432 to artery 302 comprises coupling distal cannula 432 to the selected artery.

For some applications, inserting proximal cannula 438 into right ventricle 450 comprises inserting proximal cannula 438 into a vein and advancing proximal cannula 438 through the vein, the vena cava, right atrium 114, and tricuspid valve 452, into right ventricle 450.

For some applications, coupling distal cannula 432 to artery 302 comprises anastomosing end 436 of distal cannula 432 to artery 302.

Alternatively, for some applications, RVAD 420 is implanted by inserting proximal cannula 438 of RVAD 420 through a wall of right ventricle 450.

Although some of the ventricular assist devices described herein have been described as LVADs, they may also be implemented as RVADs, mutatis mutandis.

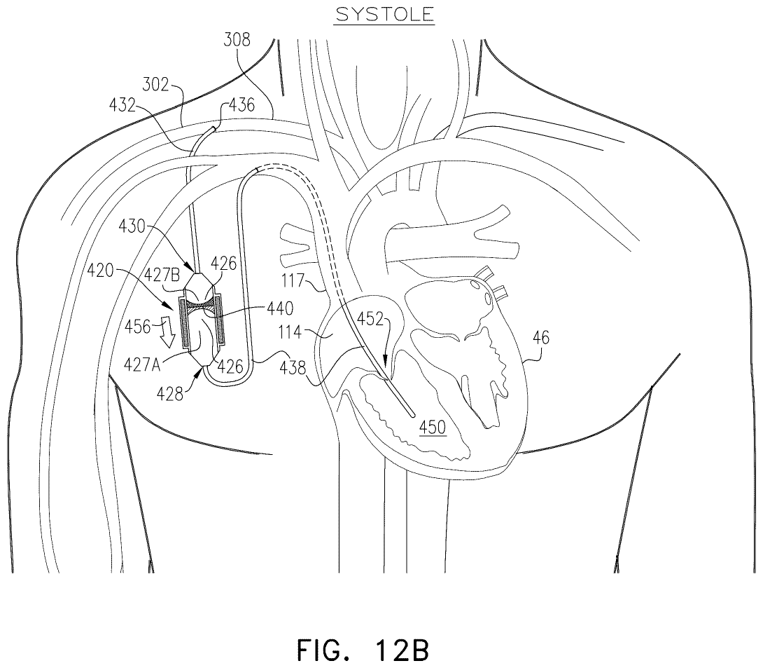

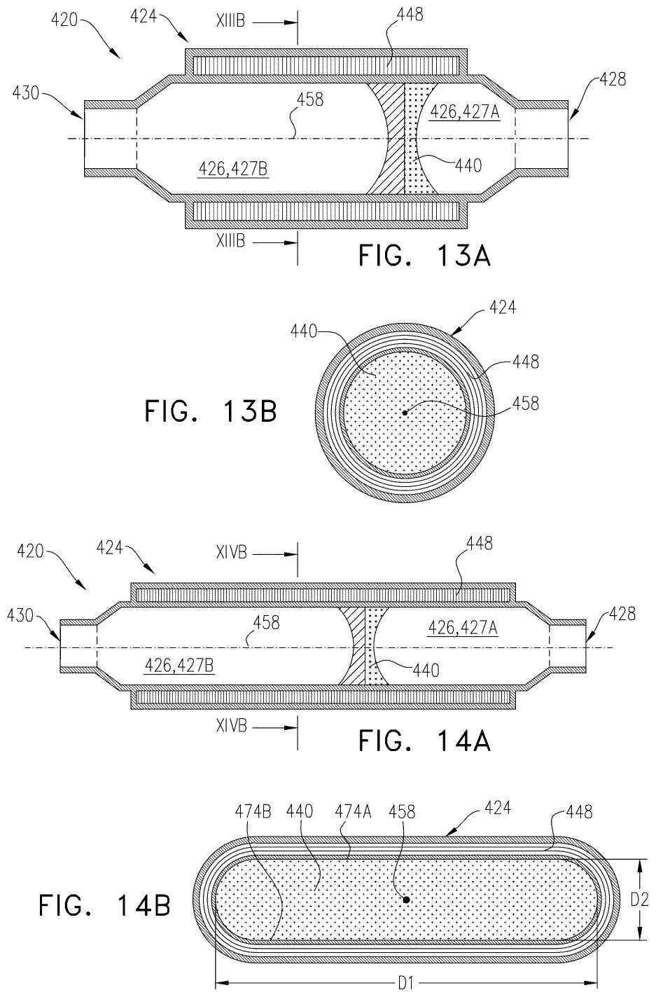

Reference is now made to FIGS. 13A-B and 14A-B, which are schematic cross-sectional illustrations of configurations of implantable RVAD 420, in accordance with respective applications of the present invention. The cross-sectional view of FIG. 13B is taken along line XIIIB-XIIIB of FIG. 13A, the cross-sectional view of FIG. 14B is taken along line XIVB-XIVB of FIG. 14A. The features of these configurations may be implemented in combination with the features of any of the other configurations of implantable RVAD 420 described herein, mutatis mutandis, and like reference numerals refer to like parts. Although magnetic piston 440 is shown in FIGS. 13A and 14A as having the shape shown in FIG. 10A, magnetic piston 440 may also have the shape described with reference to FIG. 10B.

In the configuration shown in FIGS. 13A-B, an inner surface of pump chamber 426 and an outer surface of magnetic piston 440 of pump 424 have respective circular cross-sections perpendicular to central longitudinal axis 458 of pump chamber 426.

In the configuration shown in FIGS. 14A-B, an inner surface of pump chamber 426 and an outer surface of magnetic piston 440 of pump 424 have respective elongate non-circular cross-sections perpendicular to central longitudinal axis 458 of pump chamber 426, each of which cross-sections has a first greatest dimension D1 in one direction that is greater than a second greatest dimension D2 in a perpendicular direction. For example, the first greatest dimension D1 may be at least 1.5 times the second greatest dimension D2, such as at least 2 times, e.g., at least 2.5 times the second greatest dimension D2. For example, the elongate non-circular cross-sections may be rectangular, as shown in FIG. 4B for the somewhat similar configuration; elliptical, as shown in FIG. 4C for the somewhat similar configuration; or stadium-shaped, as shown in FIG. 14B. Optionally, the cross-sections respectively include two straight portions 474A and 474B opposite each other and parallel with the first greatest dimension D1, such as shown in FIG. 14B. The elongate cross-sections provide a lower profile than the circular configuration of FIGS. 13A-B, which may facilitate subcutaneous implantation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-72. (canceled)

73. A method comprising:
inserting an inflow cannula of a left ventricular assist device (LVAD) of a LVAD system into a left chamber of a heart of a patient transeptally via a right atrium and a superior vena cava (SVC), such that the inflow cannula is positioned passing through a wall of a vein;
coupling an outflow cannula of the LVAD to an artery selected from the group consisting of: a left subclavian artery and a left axillary artery; and
implanting a pump of the LVAD in a thoracic cavity outside the heart, wherein the inflow and the outflow cannulas are coupled in fluid communication with the pump.

74. The method according to claim 73, wherein the vein is a left subclavian vein, and wherein inserting the inflow cannula comprises inserting the inflow cannula into the left chamber of the heart transeptally via the left atrium, the SVC, and the left subclavian vein, such that the inflow cannula is positioned passing through the wall of the left subclavian vein.

75. The method according to claim 73, wherein inserting the inflow cannula into the left chamber of the heart comprises inserting the inflow cannula into a left atrium such that an inflow end opening of the inflow cannula is within the left atrium.

76. The method according to claim 73, wherein inserting the inflow cannula into the left chamber of the heart comprises inserting the inflow cannula into a left ventricle via a mitral valve and a left atrium, such that an inflow end opening of the inflow cannula is within the left ventricle.

77. The method according to claim 73, wherein coupling the outflow cannula to the selected artery comprises anastomosing the outflow cannula to the selected artery.

78. The method according to claim 73, wherein the pump is a pulsatile-flow pump, and wherein the method further comprises activating control circuitry of the LVAD system to activate the pulsatile-flow pump of the LVAD to provide pulsatile flow synchronized with cardiac cycles of the patient.

79. The method according to claim 78,
wherein the pulsatile-flow pump includes a pump chamber having an upstream inlet and a downstream outlet, and includes a tubular linear motor, which includes (a) a magnetic piston, which includes a reciprocating one-way valve configured to allow downstream blood flow and inhibit upstream blood flow; and (2) a stator, which is configured to magnetically drive the magnetic piston with reciprocating motion, so as to pump blood downstream during downstream motion of the magnetic piston while the reciprocating one-way valve is closed,
wherein the outflow cannula is arranged in fluid communication with the downstream outlet of the pump chamber,
wherein the inflow cannula is arranged to allow downstream blood flow from the inflow cannula to the upstream inlet of the pump chamber, and
wherein activating the control circuitry comprises activating the control circuitry to activate the tubular linear motor to provide the pulsatile flow synchronized with the cardiac cycles by, during each of the cardiac cycles:
activating the stator of the tubular linear motor to move the magnetic piston downstream during a portion of systole of the cardiac cycle, and
activating the stator of the tubular linear motor to move the magnetic piston upstream during at least a portion of diastole of the cardiac cycle.

80. The method according to claim 79, wherein the LVAD system further includes a cardiac sensor, which is configured to sense one or more features of the cardiac cycle, and wherein the control circuitry is coupled to the cardiac sensor.

81. The method according to claim 79, wherein the LVAD system further includes a stationary one-way outflow valve, which is arranged to allow downstream blood flow from the downstream outlet of the pump chamber to the outflow cannula, and to inhibit upstream blood flow from the outflow cannula to the downstream outlet.

82-168. (canceled)

169. The method according to claim 73, wherein inserting the inflow cannula such that the inflow cannula is positioned passing through the wall of the vein comprises anastomosing the inflow cannula to the wall of the vein.
